(19) United States
(12) Patent Application Publication
(10) Pub. No.: US 2008/0293142 A1
(43) Pub. Date: Nov. 27, 2008

(54) Multiple shRNA Expression Vectors and Methods of Construction

(75) Inventors: Lin Liu, Edmond, OK (US); Deming Gou, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: The Board of Regents for Oklahoma State University, Stillwater, OK (US)

(21) Appl. No.: 12/105,428

(22) Filed: Apr. 18, 2008

Related U.S. Application Data
(60) Provisional application No. 60/912,765, filed on Apr. 19, 2007.

(51) Int. Cl. C12N 15/11

(52) U.S. Cl. 435/455; 536/24.1

(57) ABSTRACT

A research or therapeutic tool for RNA interference (RNAi) is a single vector that expresses multiple short hairpin RNA (shRNA) sequences.

### CROSS-REFERENCE TO RELATED APPLICATIONS

This application claims benefit of U.S. provisional patent application 60/912,765 filed Apr. 19, 2007, the complete contents of which is hereby incorporated by reference.

### STATEMENT REGARDING FEDERALLY SPONSORED RESEARCH

The development of the subject matter of this application was partially supported by grants from the National Institutes of Health (Grant Nos. HL-052146, HL-071628 and HL-083188). Accordingly, the U.S. government may have certain rights in this invention.

### SEQUENCE LISTING

This application includes as the Sequence Listing the complete contents of the accompanying text file “Sequence.txt”, created Apr. 8, 2008, containing 19,806 bytes, hereby incorporated by reference.

### BACKGROUND OF THE INVENTION

1. Technical Field

The present invention relates generally to the application of RNA interference (RNAi) as a research and therapeutic tool, and, more specifically, to the construction of a single vector expressing multiple short hairpin RNA (shRNA) sequences.

2. Background

The application of RNA interference (RNAi) as a research and therapeutic tool depends on its ability to silence genes in a sequence-specific manner. Recent studies have reported that the effective knockdown of genes can be achieved by multiple shRNAs in a single vector. Moreover, this approach can depress several genes simultaneously. However, current methods for the construction of multiple shRNA vectors often suffer from vector instability and the excessive consumption of time and resources in their construction.

It is accordingly an objective of the present invention to provide a simple, quick and low cost approach to construct a single stable vector expressing multiple shRNA sequences.

### SUMMARY OF THE INVENTION

The present invention provides shRNA expression cassettes that are straightforward and cost-effective to construct, and that are capable of stably expressing multiple shRNAs within a cell. Expression of the multiple shRNAs results in silencing of mRNAs within the cell in a sequence specific manner. According to the invention, transcription of at least two of the plurality of shRNAs encoded by the expression cassette is driven by two promoters that are part of a bidirectional promoter arranged in a back-to-back form, and transcription of the remaining shRNAs is driven by additional promoters present in the cassette. Typically, at least two additional promoters are present. The promoters are able to drive transcription because each promoter is operationally linked to a nucleic acid sequence that encodes an shRNA. In other words, the promoters and nucleic acid sequences are arranged with respect to each other so that transcription of each of the promoters drives transcription of one of the nucleic acid sequences encoding an shRNA. Sequence specific RNA silencing is carried out by introducing one or more of such expression cassettes into a cell in a manner that allows the shRNAs to be expressed. For example, the expression cassette may be introduced via an expression vector such as an adenoviral vector. In one embodiment of the invention, the promoters include Pol III RNA promoters. Further, the expression cassette may also include other useful sequences such as linking/spacer sequences, restriction endonuclease cleavage sites, termination signal sequences, marker sequences such as enhanced green fluorescent protein (GFP) for tracking shRNA expression in cells, etc.

The invention also includes a rapid, economical method for producing such an expression cassette. The steps of the method include 1) preparing a polymerase chain reaction (PCR) template that contains at least one bidirectional promoter comprising two promoters in a back-to-back form; and 2) amplifying by PCR the PCR template using primers that include nucleic acid sequences encoding a plurality of shRNAs. This step of amplifying produces an insert comprising 1) at least one bidirectional promoter in a back-to-back form and 2) nucleic acid sequences encoding the plurality of short hairpin RNAs. A third step of the method involves joining (e.g. by ligation) the insert to nucleic acid sequences encoding one or more additional promoters, thereby forming an expression cassette for expressing the shRNAs. Within the expression cassette, the promoters of the bidirectional promoter and the additional promoters are operationally linked to the nucleic acid sequences encoding the plurality of shRNAs. In one embodiment, the two promoters of the bidirectional promoter have a 5′ overlap, and the step of preparing is carried out by overlap PCR.

As demonstrated in the experimental results reported hereunder, a single vector expressing four shRNA sequences driven by four different promoters was constructed in a simple, quick and cost-effective method. Using this vector, we were able to improve gene silencing efficiency and make it possible to silence four different genes simultaneously, further expanding the application spectrum of RNAi, both in functional studies and therapeutic strategies. The new RNAi vector, pK4-shRNA, demonstrated high efficient suppression up to 98% of all 12 target genes tested in various cell/organ systems. Consequently, the pK4-shRNA vector eliminates the need for screening effective siRNAs and significantly lowers the dose required to achieve maximal inhibition. The inventive method of construction is well-suited for generating high-quality shRNA libraries and provides and efficient strategy for RNAi therapy.

A better understanding of the present invention, its several aspects, and its advantages will become apparent to those skilled in the art from the following detailed description, taken in conjunction with the attached figures, wherein there is described the preferred embodiment of the invention, simply by way of illustration of the best mode contemplated for carrying out the invention.

### BRIEF DESCRIPTION OF THE DRAWINGS

FIG. 1 is a schematic representation of a vector containing an expression cassette of the invention.

FIG. 2 A-C is a schematic representation of transcription of one Transcriptional Unit of the expression cassette, and the shRNA that is produced. A, a single transcription unit (Transcription Unit 1); B, transcribed ssRNA; C, base-paired shRNA.

FIG. 3 is a schematic outline for the construction of a pK4-shRNA vector. Step A, preparation of the PCR template; Step B, generation of the insert of K4-shRNA by multiple PCR amplification; Step C, Cloning the PCR inserts into the pre-made vector. Details are given in Materials and Methods.

FIG. 4A-B is a comparison of RNA pol III promoter activities. A, A 21-nt siRNA against EGFP at the position of 417˜437 in the form of a sense-loop-antisense hairpin structure (shEGFP417) was placed under the control of different RNA pol III promoters, including hU6, mU6, 7SK, H1 and a single base-mutated H1^{m} promoters. A stretch of five thymidines serves as the termination signal. B, The ability of different promoter-driven shEGFP₄₁₇ to silence EGFP in 293A cells co-transfected with pENTR/CMV-EGPF and pDsRed2-C1 (for normalization). The pENTR vector without the promoter and the shRNA sequence was used as negative control. Twenty-four hours post-transfection, cells were assayed for EGFP and DsRed2 by the FluoroMax 3 fluorometer using Ex=489 nm/Em=508 nm and Ex=563 nm/Em=582 nm, respectively. The normalized EGFP fluorescence was shown as a percentage of the control (means ±SD, n=3 replicates from one representative of 3 experiments).

FIG. 5A-E illustrates that the pK4-shRNA vector is more effective than any individual shRNA vector. A, schematic illustration of pK4-shRNA vector expressing four shEGFP against to different position of EGFP mRNA or four copies of shEGFP₄₅₀ against to the same position of EGFP at 450-470. B, comparison of the K4-shEGFP vector with the corresponding individual shRNAs. EGFP expression levels were determined 48 hrs after the co-transfection of the 293A cells with a fixed amount of the pCVM-EGFP expression plasmid (20 ng) and varying amounts of K4-shEGFP or individual shRNA vector at ratios ranging from 1:10 to 10:1. Twenty ng of pDsRed2-C1 were included to normalize the transfection efficiency. Data shown are means ±SD (n=4 independent experiments). C, comparison of pK4-EGFP containing 4 different shRNAs, pK4-shEGFP₄₅₀ containing 4 copies of the same shRNA and a mixture of 4 individual shRNA plasmids. An equal amount of pCVM-EGFP vector (20 ng) and pK4-shEGFP (20 ng), pK4-shEGFP₄₅₀ (20 ng) or the mixture of 4 individual shEGFP plasmids (total 20 ng and 5 ng each) with the normalization vector of pDsRed2-C1 vector were co-transfected into 293A cells for 2 days. Data were expressed as a percentage of the pK4-shCon containing 4 unrelated shRNAs (means ±SD, n=3 replicates from one experiment). *P<0.05 v.s. K4shCon; **P<0.05 v.s. K4-shEGFP. D and E, silencing of IGF1R (D) or SNAP-23 (E) by adenovirus-based pK4-shRNA vector was compared with each of the four individual shRNA vectors in the RLE-6NT cells at various doses. The protein level of IGF1R was determined by Western blot and normalized to β-actin. SNAP-23 mRNA was determined by real time PCR and normalized to GAPDH. Data were expressed as a percentage of blank control without virus treatment. Control virus (K-4-shCon) had no effect on the protein expression of IGF1R, or on mRNA expression of SNAP-23.

FIG. 6A-C illustrates the simultaneous knockdown of four genes by pK4-shRNA. A, schematic illustration of the four promoter-driven shRNA vector targeted to four different human genes (pK4-sh4Gene). The selected siRNA sequences were targeted to the following positions: p53, 775-793; Lamin A/C, 610-628; IGF1R, 567-588; and Bc12, 563-581. B, Northern blots showing the four shRNA transcripts. A549 cells were transduced with 100 MOI adenoviral pK4-shCon vector expressing 4 unrelated shRNA sequences (lane 1) or pK4-sh4Gene (lane 2) viral vector for 2 days. Total RNA (20 μg) was analyzed by Northern blot on a 15% polyacrylamide-urea gel. The blots were hybridized with the ³²P-labeled sense sequences of shp53, shLamin A/C, shIGF1R, or shBcl2, The same amount of 28S and 18S were observed in lanes 1 and 2. C, dose-response of silencing 4 genes by pK4-sh4Gene adenoviruses in A549 cells. A549 cells were infected using adenovirus at 100 MOI. The mRNA level was determined by real-time PCR and expressed as a percentage of the blank control without virus treatment. The results shown are means ±SD from three independent experiments.

FIG. 7A-G illustrates the specificity of pK4-shRNA. A, comparison of siRNA sequences of K4-shAIIa and K4-shRNAIIb between rat and human. B, rat lung type II cells or C, human A549 cells were transducted with pK4-shCon, pK4-shAIIa or pK4-shAIIb adenovirus. Annexin A2 protein was detected by Western blot with β-actin as a loading control. D and E, silencing of P11 (D) or SNAP-23 (E) in rat lung type II cells by adenoviral-based pK4-shP11 or pK4-shSNAP-23 expressing four siRNAs against P11 or SNAP-23 at different positions. The mRNA level of P11 was analyzed by semi-quantitative PCR with GAPDH as loading control. The protein level of SNAP-23 was detected by Western blot with GAPDH as loading control. pK4-shCon was used as control. F, cluster analysis of DNA microarray data. Primary alveolar type II cells were treated for 2 days with pK4-shAIIa, pK4-shAIIb, pK4-shSNAP-23 or pK4-p11 adenovirus at a MOI of 50 or blank control without virus treatment. Each sample was hybridized to a 10,000 rat DNA microarray with a common reference. The genes that passed the SAM test were grouped by K-means cluster analysis. Red color represents the up-regulation and green down-regulation. Annexin A2 gene was indicated by arrow. G, Venn diagrams. The numbers in each circle show the numbers of up- or down-regulated genes caused by each pK4-shRNA. The common changed genes caused by two or three pK4-shRNAs are underlined.

### DETAILED DESCRIPTION

Before explaining the present invention in detail, it is important to understand that the invention is not limited in its application to the details of the embodiments and steps described herein. The invention is capable of other embodiments and of being practiced or carried out in a variety of ways. It is to be understood that the phraseology and terminology employed herein is for the purpose of description and not of limitation.

RNA interference (RNAi) is a conserved process in which a double-stranded ˜21-nucleotide (nt) short interfering RNA (siRNA) induces the sequence-specific degradation of complementary mRNA [1]. Before RNAi can be applied to gene therapy, improvements must be made to the stability, efficiency and specificity of the chemically synthesized siRNA [2]. To overcome the transient nature of siRNA, DNA vectors have been developed to express short-hairpin RNA (shRNA) that can be converted into siRNA in vivo [3,4]. However, the efficiency and specificity of this technique is still based on the screening of the siRNA sequence. The existing rules for siRNA selection allow the identification of potential sequences, but do not ensure that each selected siRNA sequence is effective. On average, 25% of selected target siRNA sequences are functional with more than 75% knockdown efficiency [5]. Therefore, it is recommended to screen the most effective siRNA from several potential sites of a given mRNA [6-8]. To avoid such screening, a mixture of siRNAs have been generated by various methods including RNAse III or recombinant human Dicer-mediated hydrolysis of long double-stranded RNA [6-8]. The production of a shRNA expression library by enzymatically engineering cDNA has also been used [9,10]; however, an important concern is that such approaches may increase off-target effects [11].

A single DNA vector expressing multiple shRNAs against different regions of a gene is a new strategy to improve the silencing efficiency [12-15] or to knockdown several genes simultaneously [14-21]. Moreover, combined expression of multiple shRNAs could significantly delay viral escape mutants [14,15,22], indicating a promising application of multiple shRNAs in anti-viral gene therapy. An important step of this approach is the design of a DNA vector that expresses multiple shRNAs. The reported methods are based on several steps of subcloning, and thus cost and time are limiting factors [15,17,23]. In connection with the present invention, a simple and quick method is used to construct a four different pol III promoter-driven multiple shRNA expression vector, pK4-shRNA, that effectively improves the knockdown efficiency over single shRNA constructs. Evidence shows the silencing of four different genes at the same time as a result of using the vector. The application of pK4-shRNA-based gene silencing was extended to cell lines and primary cells by an adenovirus delivery system. The specificity of adenovirus-mediated pK4-shRNA vectors was also evaluated.

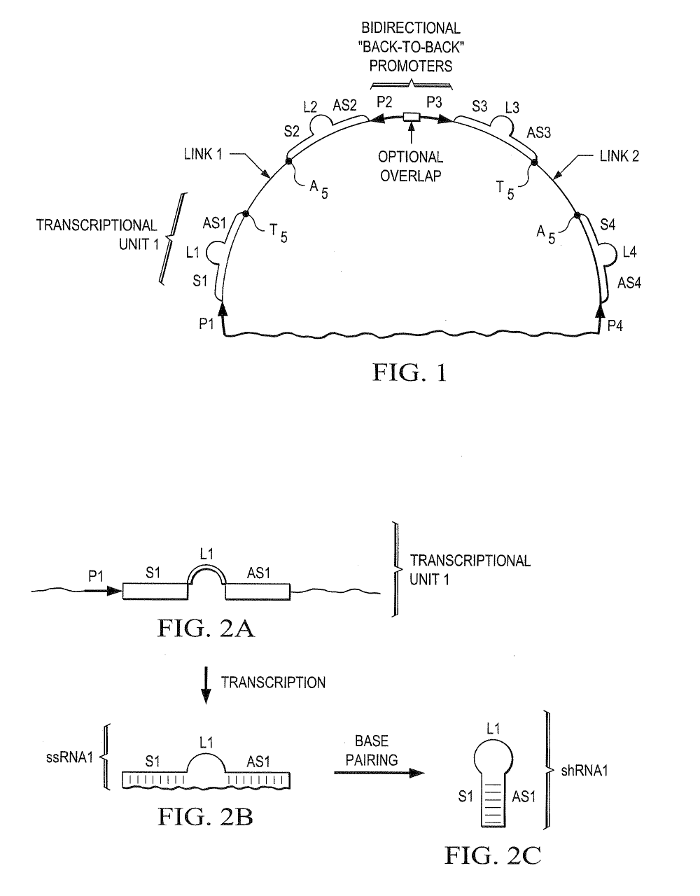

A schematic representation of an expression cassette of the invention encoding four shRNAs is presented in FIG. 1A. P2 and P3 represent the two promoters that make up the bidirectional “back-to-back” promoters from which transcription of two of the four shRNAs is driven. The two bidirectional promoter sequences may be joined, for example, by overlapping, complementary sequences (illustrated by the square labeled “optional overlap”) e.g. as a result of complementary 3′ and 5′ overhangs produced by restriction enzyme cleavage, or by simply adding the overlapping sequences during a PCR reaction, etc. However, overlap is not required. Two other promoters in the cassette are labeled P1 and P4. Thus, the exemplary expression cassette of FIG. 1A contains a total of four promoters.

While the exemplary expression cassette as depicted in FIG. 1A contains four promoters, this need not be the case. In some embodiments, only two promoters are employed, i.e. the cassette contains a single arrangement of two bidirectional promoters. Alternatively, a total of 3, 4, or even up to 8 or more promoters may be included in the cassette. In addition, more than one set of back-to-back bidirectional promoters may be in one expression cassette. Up to about 4 bidirectional promoter sets may be included in the construct. Further, the promoters in the construct may all differ from each other, or one or more of the promoters may be the same, or all of the promoters may be the same. In the exemplary construct described in the Examples below, all of the promoters are different.

Each promoter in the cassette is associated with a transcriptional unit, one of which is indicated in FIG. 2A. A single transcriptional unit comprises, at a minimum, a promoter that is operationally linked to three sequences: a sense encoding sequence, a loop encoding sequence and an antisense encoding sequence. By “sense encoding sequence” or “sense region” is meant a nucleotide sequence that encodes a portion of an shRNA molecule having complementarity to an antisense region of the same shRNA molecule. In addition, the sense region of a shRNA molecule can comprise a nucleic acid sequence having homology with a target nucleic acid sequence. By “antisense encoding sequence” or “antisense region” is meant a nucleotide sequence that encodes a portion of an shRNA molecule having complementarity to a target nucleic acid sequence. In addition, the antisense region of an shRNA molecule comprises a nucleic acid sequence having complementarity to the sense region of the shRNA molecule. By “complementarity” is meant that a nucleic acid can form hydrogen bond(s) with another nucleic acid sequence by either traditional Watson-Crick or other non-traditional types. The sense and antisense encoding sequences comprise DNA sequences that, upon transcription, produce single strand RNA sequences that are complementary to each other; whereas the transcriptional unit does not contain sequences that, upon transcription, are complementary to the loop sequence. With reference to FIG. 2A, the promoter of Transcriptional Unit 1 is labeled P1, the sequence encoding the sense sequence is labeled S1, the sequence encoding the antisense sequence is labeled AS1, and the sequence encoding the loop sequence is labeled L1. The L1 sequence is depicted with a single line and drawn as a semicircle to illustrate that a loop is encoded. However, those of skill in the art will recognize that in the cassette, all sequences are double stranded, usually DNA. In FIG. 1, a total of four transcriptional units, transcribed by promoters P1, P2, P3 and P4, are illustrated.

Generally, the sense sequence of the shRNA will be from about 19 to about 22 nucleotides (e.g. about 19, 20, 21 or 22 nucleotides) in length, the antisense sequence will be from about 19 to about 22 nucleotides (e.g. about 19, 20, 21 or 22 nucleotides), in length, and the loop region will be from about 3 to about 19 nucleotides (e.g., about 3, 4, 5, etc., . . . up to about 19) nucleotides in length. In some embodiments, the sense and antisense sequences are the same length, i.e. the shRNA will form a symmetrical hairpin, but this is not necessarily the case. In some cases, the sense or antisense strand may be shorter than its complementary strand, and an asymmetric hairpin is formed. Further, while in some instances the base pairing between the sense and antisense sequences is exact, this also need not be the case. In other words, some mismatch between the sequences may be tolerated, or even desired, e.g. to decrease the strength of the hydrogen bonding between the two strands. However, in a preferred embodiment, the sense and antisense sequences are the same length, and the base pairing between the two is exact and does not contain any mismatches. The shRNA molecule can also comprise a 5′-terminal phosphate group that can be chemically modified. In addition, the loop portion of the shRNA molecule can comprise, for example, nucleotides, non-nucleotides, linker molecules, conjugate molecules, etc.

With further reference to FIG. 2, as can be seen, transcription of Transcriptional Unit 1 results in the production of a single strand of RNA, as illustrated in FIG. 2B. The single strand of RNA contains the sense RNA (S1) and the complementary antisense RNA (AS1), with the loop encoding RNA (L1) interposed therebetween. Since there is no nucleic acid to complement the loop sequence, when base pairing takes place between S1 and AS1, a “short hairpin” RNA (shRNA) structure with a single strand loop (L1) is produced, as depicted schematically in FIG. 2C.

The several transcriptional units that are included in an expression cassette of the invention may each encode a different shRNA, or they may all encode the same identical shRNA, or some may encode the same shRNA while others encode different shRNAs. In addition, the shRNAs may target different regions of a single mRNA molecule. Both coding or non-coding regions may be targeted. Further, in embodiments of the invention in which a single sequence is targeted, but for which the ideal inhibitory shRNA is not known, the cassette may encode several shRNAs that are highly homologous but have differences intended to span several variant sequences that are deemed most likely to effectively bind to and inhibit the target RNA, either at a single location, at overlapping locations, or at different locations along the RNA molecule. As explained herein, encoding several of such variants on a single construct eliminates the need to make multiple constructs and test each one individually to optimize results. By “highly homologous” we mean that the nucleotide sequences of the variants are either identical or perfectly complementary, or are the same or complementary over at least about 50, 60, 70, 80, or 90% of their sequences, and preferably about 91, 92, 93, 94, 95, 96, 97, 98, or 99% homologous. Those of skill in the art are familiar with calculating the homology of nucleic acids and any suitable method may be utilized. For example, sequences that hybridize under conditions of high stringency are typically considered to be highly homologous. Alternatively, one may simply count the bases and determine mathematically how many are the same and how many differ between two strands that are being compared. For example, a percent complementarity may indicate the percentage of contiguous residues in a nucleic acid molecule that can form hydrogen bonds (e.g., Watson-Crick base pairing) with a second nucleic acid sequence, e.g., 5, 6, 7, 8, 9, or nucleotides out of a total of 10 nucleotides in the first oligonucleotide being based paired to a second nucleic acid sequence having 10 nucleotides represents 50%, 60%, 70%, 80%, 90%, and 100% complementary respectively. “Perfectly complementary” means that all the contiguous residues of a nucleic acid sequence will hydrogen bond with the same number of contiguous residues in a second nucleic acid sequence.

Those of skill in the art will recognize that many different promoters exist that may be employed in the practice of the invention, examples of which include but are not limited to the following:

Tissue or cell-specific promoters such as the following, which are listed with the cells or tissues for which they are specific:

SP-C and SP-B promoter: lung epithelial type II cells
Aquaporin 5 promoter; lung epithelial type I cells
CCSP promoter; lung Clara cells
Cytokeratin 18 (K18) promoter; lung epithelial cells
Vascular endothelial growth factor receptor type-1 (flt-1) promoter: endothelial cells
FOXJI promoter; lung airway surface epithelium.
Tie2 promoter, lung endothelial cells
Pre-proendothelin-1 (PPE-1) promoter, endothelial cells
Albumin promoter, liver
MCK promoter, muscle
Myelin basic protein promoter, oligodendrocytes glial cells
Glial fibrillary acidic protein promoter, glial cells
NSE promoter, neurons
KDR, E-selectin, and Endoglin promoters, tumor endothelium
Telomerase reverse transcriptase promoter; cancer cells.
Carcinoembryonic antigen (CEA) promoter; lung, breast, colon cancers
Alpha-ftoprotein (AFP) promoter; hepatocellular carcinoma (HCC)
ErbB2 promoter, breast cancer
Tyrosinase gene promoter, melanoma
Prostate-specific antigen (PSA) promoter, prostate-specific
Muc-1 promoter, breast cancer
Osteocalcin promoter, osteosarcoma
Secretory leukoprotease inhibitor, ovarian, cervical carcinoma
HRE promoter, solid tumours

In other embodiments of the invention, inducible promoters may be used, examples of which include but are not limited to: (1) tetracycline-inducible system: The shRNA expression is under the control of the modified U6, H1, or 7SK promoter, in which the tetracycline operator (TetO) sequence is added. The tetracycline repressor (tTR) or tTR-KRAB expression is under the control of cell-specific promoter, such as SP-C promoter. In the absence of an inducer, the tTR or t-TR-KRAB binds to TetO and inhibits the expression of shRNA. The addition the inducer, doxycycline (DOX) removes the tTR or tTR-KRAB from the TetO and thus induces the transcription of shRNA in a cell-dependent manner since tTR or tTR-KRAB is only expressed in a specific cell type. (2) IPTG-inducible system. This is similar to (1) above except that TetO and tTR are replaced with lac operator and lac repressor, respectively. The inducer in this case is isopropyl-thio-beta-D-galactopyranoside (IPTG). (3) CER inducible system: a neomycin cassette (neo) is inserted into the U6 or H1 promoter that drives shRNA expression. The insertion disrupts the promoter activity and thus no transcription of shRNA occurs. However, the cell-specific expression of Cre recombinase under the control of a cell-specific promoter restores the promoter activity and thus the expression of shRNA in a specific cell type. The inducer in this case is tamoxifen. (4) Ecdysone-inducible system. The inducible ecdysone-responsive element/Hsmin (ERE/Hsmin) is added to U6 promoter that controls the expression of shRNA. The expression of two proteins, VgEcR and RXR are driven by cell-specific promoters. In the presence of the inducer, MurA, VgEcR and RXR form a dimer and bind to ERS/Hsmin to initiate the transcription of shRNA in a specific cell type. It will be understood that a construct can have more than one constitutive promoter, as well as combinations of constitutive and inducible promoters.

Other promoters that may be utilized include but are not limited the SV40 early promoter, the cytomegalovirus immediate early promoter/enhancer and the rous sarcoma virus long terminal repeat promoters; or the eukaryotic promoters or parts thereof, such as the β-casein, uteroglobin, β-actin, ubiquitin or tyrosinase promoters. Any known promoter sequence may be utilized, so long as it is susceptible to insertion into the cassette, and can be operationally linked to the sequences encoding the shRNA, i.e. so long as it causes transcription of the sequences that make up the shRNA. In some embodiments, the mU6, hU6, 7SK and H1^{m} promoters are employed.

The expression cassettes of the invention also contain linker sequences between the transcriptional units for which transcription is not driven by the bidirectional promoters, and/or between transcriptional units that include a bidirectional promoter and those that do not (e.g. Link 1 and Link 2 in FIG. 1). Such linker or spacer sequences serve as “linkers” for overlap PCR and to separate each transcriptional unit or bidirectional promoters. Exemplary linker sequences are from about 10 to about 17 nucleotides in length. Examples of the suitable linker sequences include but are not limited to: 5′-GACCTTGGATCGATCCG-3′ (SEQ ID NO: 105); 5′-GCTCAGCGGAG-3′ (SEQ ID NO: 106); 5′-TTCAGTCCGAG-3′ (SEQ ID NO: 107).

In addition, in some embodiments of the invention, the linker sequences in the expression cassette are flanked by sequences that encode a transcription termination signal i.e. a “run” or “string” of thymine (T) nucleotides. In one embodiment, each linker is flanked by from about 5 to about 7 T residues on both sides. In the exemplary expression cassette depicted in FIG. 1, Link 1 is flanked by five T's on the 5′ end of the linker (which abuts Transcriptional Unit 1), and by five A's on the 3′ end of the linker (adjacent to Transcriptional Unit 2, which includes P2 of the bidirectional promoter plus AS2, L2 and S2). The former is represented by T's and the latter is represented by A's because they are both double strand DNA, and the direction of transcription for the two is opposite.

The generation of multiple shRNAs from a single expression cassette as described herein is economical, both in terms of the amount of time and labor that is involved, and in the resulting cassette that can be used to express a plurality of shRNAs at once. As described above, this is advantageous in many situations where it is preferable to silence more that one mRNA, or to increase the probability of silencing one mRNA by providing several variant shRNAs, some of which may work with greater efficacy than others. Rather than constructing multiple expression cassettes and testing one at a time, a single cassette can be constructed to produce multiple shRNAs.

The shRNAs produced by the methods of the invention are typically directed against one or more target RNAs. By “target RNA” is meant any RNA sequence, usually within a cell, whose expression or activity is to be modulated, usually inhibited, downregulated or reduced. By “inhibit”, “down-regulate”, or “reduce”, it is meant that translation of mRNA molecules encoding one or more proteins or protein subunits is reduced below that observed in the absence of the shRNA molecules of the invention. In one embodiment, inhibition, down-regulation or reduction with an shRNA molecule refers to translation of the mRNA that is below a level observed in the presence of an inactive or attenuated mRNA molecule, or inactive or attenuated peptide, polypeptide or protein encoded by the mRNA. In another embodiment, inhibition, down-regulation, or reduction with shRNA molecules refers to translation of the mRNA at a level observed in the presence of, for example, an shRNA molecule with scrambled sequences, mismatches, etc., that render the shRNA non-complementary to the target RNA. In preferred embodiments, the target RNA is mRNA, however other types of RNA (e.g. non-coding RNAs such as rRNA and tRNA, as well as microRNA transcripts) may also be targeted.

In general, the purpose of targeting an mRNA sequence is to destroy the sequence and prevent its translation, particularly in a biological system such as within a cell. One advantage of the expression cassettes and vectors of the invention is that they are stable in the intracellular environment. By “stable” we mean that, once inside a living cell, the expression cassette or vector (usually double strand DNA) will persist in an active, useful form i.e. a form from which shRNA may be transcribed, for a period of time ranging from several days to permanent transcription, e.g. is a lentivirus or other vector that has the ability to integrate a transgene into the host genome is used, and a stable cell line is established.

The result of preventing the translation of a target RNA is intended to have a beneficial effect on the cell. For example, the result may be slowed growth or death of a cell, e.g. cancer cells or other undesirable cells such as disease causing agents, parasites, cells infected by viruses, etc. This typically comes about because the shRNA prevents translation of the mRNA that encodes a peptide, polypeptide or protein that is necessary for the cell to survive, or to replicate, etc. Alternatively, the result may be increased expression of a beneficial protein, e.g. by destroying mRNA that encodes an inhibitor of the protein; etc. Those of skill in the art will recognize a plethora of different applications of the technology described herein. Further examples of the use of siRNAs in general, and shRNAs in particular, are discussed, for example, in U.S. Pat. No. 7,067,249 to Kung et al. and U.S. Pat. No. 7,176,304 to McSwiggen et al., the contents of both of which are hereby incorporated by reference.

The shRNAs that are generated from the expression cassettes of the invention may be administered to a cell or cells of interest in any of several different ways. The shRNAs may be conveniently made in vitro and administered as shRNA according to methods known in the art. Alternatively, the shRNA may be transcribed in vivo, within the cell or cells of interest. In this case, the expression cassettes of the invention (usually double strand DNA) may be administered directly to the cell or cells by methods known to those of skill in the art, e.g. by using a solution that permeates the cell membrane, complexed with cationic lipids, packaged within liposomes, by electroporation, transfection, or otherwise delivered to target cells or tissues. The nucleic acid or nucleic acid complexes can be locally administered to relevant tissues ex vivo, or in vivo through injection, infusion pump or stent, with or without their incorporation in biopolymers, etc.

Alternatively, the expression cassettes may be inserted into a suitable vector (usually a double strand DNA vector) prior to use, and such vectors are also encompassed by the present invention. In this case, the shRNAs are transcribed within the cell or cells of interest after administration of the vector to the cell or cells. By “vector” is meant any nucleic acid- and/or viral-based construct used to deliver a desired nucleic acid. Suitable vectors for administering the cassette include but are not limited to various virus-based vector such as adenoviral, lentiviral, adeno-associated viral, retroviral vectors, various plasmid-based vectors and other vectors such as baculovirus, phage, phagemids, cosmids, phosmids, bacterial artificial chromosomes, P1-based artificial chromosomes, yeast plasmids, and yeast artificial chromosomes etc. Delivery of shRNA expressing vectors can be systemic, such as by intravenous or intramuscular administration, by administration to target cells ex-planted from a subject followed by reintroduction into the subject, or by any other means that would allow for introduction into the desired target cell(s) or tissue, for example, transduction. In addition, those of skill in the art will recognize that some vectors may not be suitable for administration to animals, but highly suitable for storage or manipulation of the cassette, or for administration in a laboratory setting, e.g. to suppress mRNA translation in bacteria, parasites, or other organisms of interest. In one embodiment of the invention, the vector is pK4-shRNA as described in the Examples section below.

FIG. 1 depicts a vector of the invention, where the portion of the double-strand DNA that includes the expression cassette as described herein is bounded by P1 and P4. The “wavy” line between P1 and P4 represents the portion of the vector that is not part of the cassette per se. This portion of the vector may encode a wide variety of different entities that include but are not limited to, for example: the elements of an adenoviral (AD) vector that are necessary for AD vector replication; various markers or labels such as Green Fluorescent Protein (GFP), LacZ, or red fluorescent protein; and/or various genes that encode proteins that it is desirable to express along with the shRNAs of the invention.

The invention also encompasses compositions for delivering the constructs of the invention to cells of interest. Those of skill in the art are knowledgeable concerning such compositions. In particular, when the composition is used pharmaceutically, the composition may contain e.g. a physiologically compatible carrier such as saline, phosphate buffered saline, etc. In general, such compositions may include various additives, preservatives, diluents, thickeners, salts, buffers, and the like, suited to the form of administration.

The cells of interest to which the expression cassettes of the invention are administered include but are not limited to, for example, any type of in vitro cell such as various cultured cell lines; cells from primary cell culture; single celled prokaryotes; lower eukaryotic organisms; etc. In such cases, the expression cassettes may, for example, be used as a valuable research tool.

In other embodiments of the invention, the constructs of the invention (i.e. the expression cassettes, the shRNAs produced by them, or vectors in which the expression cassettes are housed) are administered to multicellular organisms or to particular subsets of cells within multicellular organisms such as animals (e.g. to a particular organ or tissue). The target RNA can be mRNA that is encoded by a gene that is endogenous to the cell, or encoded by a transgene, or encoded by exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates (including humans) and invertebrates. Non-limiting examples of fungi include molds and yeasts. Of special interest is administration to human patients who may benefit from the expression of the shRNAs encoded by the cassettes to treat a disease condition that can be ameliorated by the inhibition of the activity of one or more RNA molecules. Examples of such disease conditions include but are not limited to

Cancer, e.g. lung cancer, leukemia and lymphoma, pancreatic cancer, colon cancer, prostate cancer, glioblastoma, ovarian cancer, breast cancer, head and neck cancer, liver cancer, skin cancer, uterine cancer; for which potential target genes (i.e. genes in the cell or tissue type that will be silenced) include: BCR/ABL fusion protein, K-RAS, H-RAS, bcl-2, Bax, FGF-4, Skp-2, CEACAM6, MMP-9, Rho, spingosine-1 phosphate-R, EGF receptor, EphA2, focal adhesion kinase, survivin, colony-stimulating factor, Wnt, PI3 kinase, Cox-2, H-Ras, CXCR4, BRAF, Brk, PKC-alpha, telomerase, myc, ErbB-2, cyclin D1, TGF-alpha, Akt-2,3, a6b4 integrin, EPCAM receptor, androgen receptor, and MDR.

Infectious diseases, e.g. HIV, Hepatitis B and C, Respiratory syncytial virus, inflenza, West Nile virus, Coxsakievirus, severe acute respiratory syndrome (SARS), cytomeglovirus, Paillomomavirus, poliovirus, Rous sarcoma virus, Rotavirus, Adenovirus, Rhinovirus, Poliovirus, Malaria (parasites); for which potential target genes include: viral genes or host receptors (CCR5, CD4, HB surface antigen, viral genes, CD46, PP1).

Ocular diseases, e.g. age-related macular degeneration, herpetic stromal keratitis, diabetic retinopathy; for which potential target genes include: VEGF, VEGF receptor, and TGF-beta receptor.

Neurological diseases e.g. amyotrophic lateral sclerosis, Alzheimer's disease, myastenic disorders, Huntingon's disease, Spinocerebellar ataxia; for which potential target genes include: SOD1, Beta-secretase (BACE1), SCCMS, Huntingin, Ataxin 1. Respiratory diseases, e.g. asthma, chronic obstructive pulmonary diseases (COPD), cystic fibrosis, acute lung injury; for which potential target genes include: TGF-alpha, TGF-beta, Smad, CFTR, MIP-2, keratinocyte-derived chemokine (KC). Other conditions or disorders, e.g. Metabolism diseases (obesity, cholesterol), inflammation (Rheumatoid arthritis), Hearing (autosomal dominant) etc; for which potential target genes include: AGRP, Apo B, TNF-alpha, Gap junction beta2.

The invention also provides a method of preparing an expression cassette for expressing a plurality of shRNAs. The method includes the steps of preparing a polymerase chain reaction (PCR) template containing at least one bidirectional promoter in a back-to-back form. This PCR template is then amplified using primers that include nucleic acid sequences which encode the plurality of shRNAs. Of note, if the two promoters of a bidirectional promoter have a 5′ overlap, then preparation of the PCR template may be carried out by overlap PCR.

The step of amplifying produces an insert that includes 1) the at least one bidirectional promoter in a back-to-back form and 2) nucleic acid sequences encoding the short hairpin RNAs. Next, the insert is joined to nucleic acid sequences encoding one or more additional promoters, thereby forming an expression cassette for expressing the shRNAs. The joining of the insert and the additional promoters may be carried out by a ligation reaction.

It is noted that, in the expression cassette, the promoters (both those of the bidirectional promoter and the additional promoters) are operationally linked to the nucleic acid sequences encoding the various shRNAs that are encoded. Each promoter is linked to one such sequence. In other words, the promoters are situated or placed with respect to the sequences encoding the shRNAs in a manner that permits, allows or even induces the promoters to carry out transcription of those sequences into shRNA under conditions in which the promoters are active. Such conditions (e.g. suitable temperature and pH, presence of various factors that cause promoters to function, suitable reservoir of ribonucleotides to incorporate into the shRNA, etc.) are well known to those of skill in the art, generally occur naturally within most viable living cells, and can be reproduced, e.g. in in vitro translation systems. This arrangement is also sometimes referred to as the promoters being “expressibly linked” to the nucleic acid sequences (or vice versa). Alternatively, the nucleic acids sequences may be referred to as “expressible” or “transcribable” or even “capable of being transcribed” (in this case into shRNA) by the promoter.

The present invention will be further understood with reference to the following non-limiting experimental examples.

### EXAMPLES

### Materials and Methods

Generation of the pK4-shRNA Vector

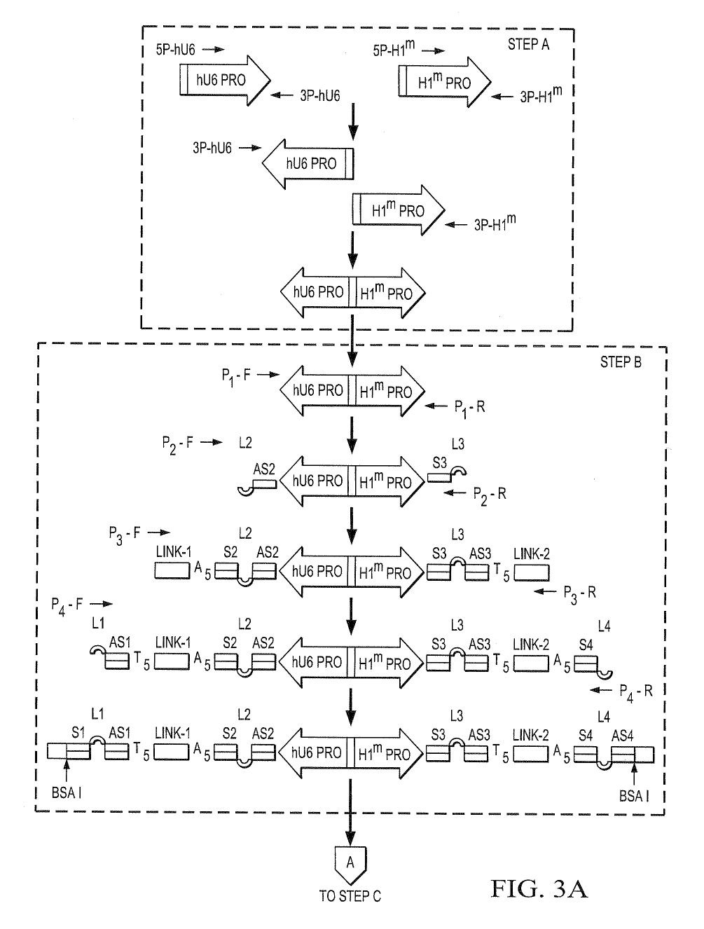

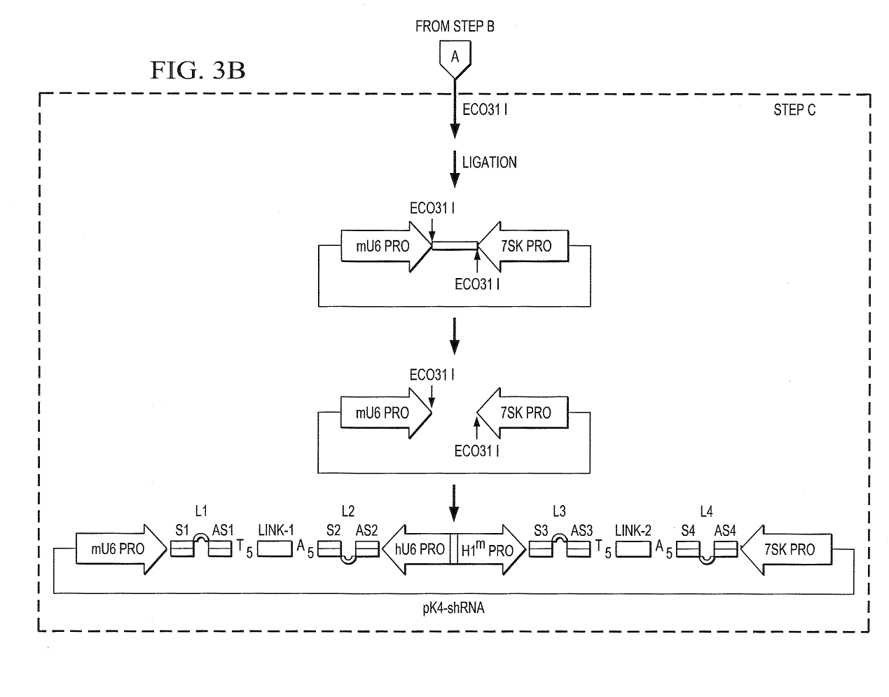

The pK4-shRNA vector containing 4 shRNAs driven by 4 different promoters was constructed by the following 3 steps (FIG. 3).

(A) Generation of the hU6-H1^{m} Template for PCR Amplification

hU6 and H1^{m} promoters were amplified from human genomic DNA (prepared from a human kidney cell line, 293A) with pfu polymerase (Stratagene) and primer sets, 5P-hU6 (5′-CGGATCGATCCAAGGTCGGGCAGGAAGAGG-3′) (SEQ ID NO:1) and 3P-hU6 (5′-GGTGTTTCGTCCTTTCCA-3′) (SEQ ID NO:2) for hU6, 5P-H1^{m} (5′-GACCTTGGATCGATCCGAACGCTGACGTCATCAACC-3′) (SEQ ID NO:3) and 3P-H1^{m} (5′-GGGGATCTGTGATCTCATACAGAACTTATA-3′) (SEQ ID NO:4) for H1^{m}. For the purpose of the subsequent cloning, a mutated base (underlined, from G to A) was introduced into the 3P-H1 primer to destroy the recognition sequence (GGTCTC) of the Eco31 I restriction enzyme. The mutated H1 promoter (H1^{m}) has similar silencing activity as the wild type (FIG. 2). Based on the 17-nt overlap (GACCTTGGATCGATCCG) (SEQ ID NO:5) between these two promoters at their 5′-end, a bi-directional hU6-H1^{m} promoter in a back-to-back form was generated by overlap PCR with the purified two promoter mixtures as the template and 3P-hU6 and 3P-H1^{m} as primers. The resulting hU6-H1^{m} PCR product (0.5 kb) was purified and used as a template to generate the insert of the pK4-shRNA vector in step B.

(B) Generation of the Insert for the pK4-shRNA Vector by 4-Step PCR Amplification

To prepare PCR fragments containing two promoters, four shRNAs and the cloning sites, we designed four sets of primers using an in-house written Excel-based program, K4-PRIMER. The siRNA sequences were designed by the web-based SiRNA Design Software (SDS) (25). SDS is a unified platform that helps to design siRNA sequences by using combination of 13 existing siRNA design software. It also filters ineffective siRNAs based on secondary structures. The software ranks each of the identified siRNA sequences based on the number of software that pick up the same sequence. We selected the highest rank of siRNA sequences. We eliminated the sequences with Eco31 I restriction site for the cloning propose (see FIG. 1). Additionally, we performed Blast search (26) to ensure the selected sequences that are specific for the gene of interest and show no significant homology to other genes. Four siRNA sense sequences (s1, s2, s3 and s4) were input into the K4-PRIMER and eight primers (P₁-F, P₁-R, P₂-F, P₂-R, P₃-F, P₃-R, P₄-F and P₄-R) were automatically generated with the following rules: from 5′ to 3′, P₁-F: 11-nt loop 2 (L2,5′-GGACAGCACAC-3′) (SEQ ID NO:6), the second siRNA antisense (as2) and a 18-nt sequence (5′-GGTGTTTCGTCCTTTC-3′) (SEQ ID NO:7) complementary to the 3′-end of the hU6 promoter; P₁-R: the last three bases of third sense siRNA, 9-nt loop 3 (L3,5′-TCTCTTGAA-3′), the third siRNA antisense sequence (as3) and a 15-nt sequence (5′-GGGAAAGAGTGATC-3′) (SEQ ID NO:8) complementary to the 3′-end of H1^{m} promoter; P₂-F: a Link-1 sequence (5′-GCTCAGCGGAG-3′) (SEQ ID NO:9), a stretch of five deoxyadenosines (A₅), the second siRNA sense (s2) and L2 sequences; P₂-R: a Link-2 sequence (5′-TTCAGTCCGAG-3′) (SEQ ID NO:10), A₅, s3 and L3; P₃-F: a 10-nt loop 1 (L1, 5′-CTTCCTGTCA-3′) (SEQ ID NO:11), the first siRNA antisense sequences (as1), T5 and Link-1 sequences; P₃-R: the last two bases of s4, a 10-nt loop 4 (L4,5′-TTGATATCCG-3′) (SEQ ID NO:12), the fourth siRNA antisense (as4), T5 and Link-2 sequences; P₄-F: a universal sequence (5′-GCATTCACGGTCTCATTTG-3′) (SEQ ID NO: 13) containing a Eco31 I restriction site, the first siRNA sense sequence (s1) and L1; P₄-R: a universal sequence (5′-GCAGTAACGGTCTCTCCTC-3′) (SEQ ID NO:14) containing another Eco31 I site, s4 and L4 sequences. All of the primers were less that 50-nt in size and synthesized by Sigma Genosys. The first step PCR was amplified by P₁-F and P₁-R using Advance 2 Taq polymerase (Clontech) and hU6-H1^{m} as a template. Ten μl of PCR products were separated on agarose gel. The single band was cut and dissolved in 50 μl 1×TE buffer, frozen at −80° C. for 20 min and then kept at 72° C. for 20 min. After centrifugation at 1,400 rpm for 5 min, one μl of supernatant was directly used as a template in the second PCR with P₂-F and P₂-R primers. This procedure was repeated for the third and fourth PCR using P₃-F/P₃-R and P₄-F/P₄-R primers. The PCR conditions were as follows: heat to 95° C. for 2 min; 2 cycles of: 95° C. for 30 sec, 60° C. for 30 sec and 68° C. for 1 min; 25 cycles of: 95° C. for 30 sec and 68° C. for 1 min; a final elongation for 7 min. The reaction volume was 15 μl for the first three PCR amplifications, but increased to 50 μl for the last step of the PCR in order to obtain enough amounts of the final K4-PCR products for digestion and ligation.

### (C) Digestion and Ligation

To prepare the pK4-shRNA expression vector, we first generated a pmU6-7SK vector containing mU6 and 7SK promoters and two Eco31 I sites. We amplified the mU6 promoter from the pSilencer 1.0 vector using primers 5′-CACCGCGGATCGATCCGACGCCGCCATCTCTA-3′ (SEQ ID NO:15) and 5′-CTTCGAAGAATTCCCGGGTCT CAAACAAGGCTTTTCTCCAA-3′ (SEQ ID NO:16) and directly cloned the PCR products into the pENTR/D-Topo vector (Invitrogen), resulting in a pmU6 vector. Four restriction sites, including BstB I, EcoR I, Sma I, and Eco31 I were introduced at the 3′-end of the mU6 promoter. Another promoter, 7SK was amplified from human genomic DNA, using the primers 5′-CTTCGAAGGTACCTGCAGTATTTAGCATGCCCCACCCATC-3′ (SEQ ID NO:17) and 5′-GGAATTCGGTCTCTGAGGTACCCAGGCGGCGCACAAGC-3′ (SEQ ID NO:18). The BstB I and EcoR I double-digested 7SK promoter was sub-cloned into the pmU6 vector through the corresponding sites, resulting in a new vector, pmU6-7SK. Two tandem Eco31 I sites were created just downstream from the mU6 and 7SK promoters. For the convenience of tracking shRNA expression, a CMV-driven EGFP expression cassette was inserted into the pmU6-7SK vector through BstB I-Asc I sites located at the upstream of 7SK promoter. This resulted in another ready-to-use vector, pEGFP/mU6-7SK. Digestion of pmU6-7Sk or pEGFP/mU6-7SK vectors with Eco31 I (Fermentas, 37° C.) left CAAA and GAGG 5′-overhangs that were then ligated to the 5′-TTTG and 5′-CCTC overhangs of the Eco31 I-digested K4-PCR products. The ligation reaction mixture was transformed into GT116 bacteria (InvivoGen) and plasmid DNA was prepared with a Qiagen miniprep kit.

### Adenovirus Generation

shRNA expression cassettes with or without an EGFP reporter gene in the pENTR/D-Topo vector were switched into an adenoviral vector, pAd/PL-DEST, through the Gateway technique (Invitrogen). Pac I-linearized adenoviral plasmids were transfected into 293A cells to generate the adenovirus. Eight to ten days after transfection, the recombinant virus was collected and subjected to one-round of amplification in a 100-mm culture dish using 3×10⁶ 293A cells. This resulted in 8 to 9 ml of viral stocks. The viral titers were determined in transduced 293A cells through EGFP expression or with the Adeno-X™ Rapid Titer Kit (Clontech).

### Cell Culture and DNA Transfection or Adenovirus Transduction

A 293A cell line, a permanent line established from human embryonic kidney cells transformed by sheared human Adenovirus type 5 DNA, was purchased from Invitrogen and cultured in DMEM medium with 10% FBS. RLE-6NT (a rat alveolar type II cell line), L2 (a rat lung epithelial cell line), and A549 (a human lung epithelial cell line) were purchased from ATCC and maintained according to the manufacturer's protocols. Primary alveolar type II cells were isolated from the perfused lungs of male Sprague-Dawley rats and cultured on an air-liquid model as previously described [24].

Transfection was performed with the appropriate plasmids using LipofectAMINE 2000 (Invitrogen). The efficiencies were evaluated according to the percentage of EGFP positive cells. The plasmid transfection efficiency in 293A cells was over 85% with a cell viability of >95% as measured by MTT assay. For adenovirus-based shRNA delivery, the transduction of adenovirus at a multiplicity of infection (MOI) of 100 led to almost all of cells infected with a cell viability of >90%.

### RNAi EGFP Suppression Assays

The 293A cells were cultured in 96-well plates until >80% confluence was obtained. The cells were transfected with 20 ng of the target pCMV-EGFP plasmid and an appropriate amount of the shRNA expression vector by using Lipfectamine 2000 reagent. To normalize the transfection efficiency, 20 ng of a red fluorescent protein reporter plasmid (pDsRed2-C1 vector, Clontech) was co-transfected into 293A cells. After 48 h, the cells were washed twice with phosphate-buffered saline. One hundred μl of lysis buffer (40 mM Hepes, pH 7.0, 100 mM KCl, 1 mM EGTA, 2 mM MgCl₂, and a protease inhibitor cocktail including 1 mM PMSF, 10 μg/ml leupeptin, 1 μg/ml aprotinin, 1 μg/ml benzamidine, and 10 uM pepstatin) were added to each well, and the cells were freeze-thawed 3 times. After centrifugation for 10 min, 5 μl of the supernatant was used to measure the expression of EGFP and DsRed2, which was determined by the FluoroMax 3 fluorometer using Ex=489 nm/Em=508 nm and Ex=564 nm/Em=585 nm, respectively.

### Real-Time PCR

Total RNA was purified with TRI Reagent (Molecular Research Center, Inc). The cDNA was synthesized with MLV reverse transcriptase. Real-time PCR was performed on an ABI Prism 7500 with QuantiTech SYBR green PCR kit (Qiagen). The primers used were: 5′-GCAGCATCCTAGGGAACCTAAAG-3′ (SEQ ID NO:19) and 5′-TGCTCTTGTATTGGCAATGTCAA-3′ (SEQ ID NO:20) for rat SNAP-23; 5′-ACCTCACCAACCCAAACACTGTA-3′ (SEQ ID NO:21) and 5′-ACATTCTCTCCCGTTTTTGCACT-3′ (SEQ ID NO:22) for rat rab14; 5′-AGTGCTCATGGAAAGGGAGTTC-3′ (SEQ ID NO:23) and 5′-AAAGCTCTGGAAGCCCACTTTT (SEQ ID NO:24) for rat p11; 5′-TGAATGAGGCCTTGGAACTCA-3′ (SEQ ID NO:25) and 5-CAGGCCCTTCTGTCTTGAACAT-3′ (SEQ ID NO:26) for human p53; 5′-CCTACCGACCTGGTGTGGAA-3′ (SEQ ID NO:27) and 5′-CTCGTCGTCCTCAACCACAGT-3′ (SEQ ID NO:28) for human Lamin A/C; 5′-GGATGTCTCCTGAGTCCCTCAA-3′ (SEQ ID NO:29) and 5′-AAGGACTTGCTCGTTGGACAA-3′ (SEQ ID NO:30) for human IGFIR; 5′-CATGTGTGTGGAGAGCGTCAA-3′ (SEQ ID NO: 31) and 5′-CTACCCAGCCTCCGTTATCCT-3′ (SEQ ID NO:32) for human Bcl2; 5′-AACTCCCTCAAGATTGTCAGCAA-3′ (SEQ ID NO:33) and 5′-CACAGTCTTCTGAGTGGCAGTGA-3′ (SEQ ID NO:34) for rat GAPDH; and 5′-AACAGCCTCAAGATCATCAGCAA-3′ (SEQ ID NO:35) and 5′-CACAGTCTTCTGGGTGGCAGTGA-3′ (SEQ ID NO:36) for human GAPDH. Data were normalized to GADPH.

### Northern Blot

A549 cells cultured overnight in 100-mm plates were transduced using pK4-sh4Gene adenovirus, which expressed four shRNAs targeted to 4 different human genes, p53 (775 to 793), Lamin A/C (610 to 628), IGFIR (567 to 588) and Bcl2 (563 to 581) or a pK4-shCon adenovirus control, which expressed 4 unrelated siRNAs: 5′AATTCTCCGAACGTGTCACGT-3′ (SEQ ID NO:37); 5′GACAGCTAGGTTATCACGATC-3′ (SEQ ID NO:38); 5′TGCGTTAGCTGCGTCAAGCAT-3′ (SEQ ID NO:39) and 5′ACTTACTGTGCGTAGTTAGCC-3′ (SEQ ID NO:40) at 100 MOI. Total RNA was isolated with TRI reagents after a 2-day transduction. RNA (20 μg) was separated on a 15% PAGE gel, electroblotted to a Hybond N⁺ membrane and UV cross-linked. The sense strand (25 pmoles) of the p53-, Lamin A/C-, IGF1R-, and Bcl2-siRNA were end-labeled with polynucleotide kinase and [³²P]-ATP (150 μCi), purified through a G-25 MicroSpin Column, heated for 5 min at 65° C., and hybridized at 37° C. overnight. Blots were washed at room temperature 2×5 min in 2×SSC plus 0.1% SDS, 3×10 min in 0.1×SSC plus 0.1% SDS, and exposed to a X-ray film.

### Western Blot

Equal numbers of cells were lysed in the SDS sample buffer, boiled and loaded onto 8-12% SDS PAGE gels. Western blotting was performed using the following primary and secondary antibodies: anti-Annexin A2 (Santa Cruz, 1:1,000), anti-SNAP-23 (Synaptic Systems, 1:1000), anti-Smad4 (Santa Cruz, 1:2,000), anti-IGF1R (Santa Cruz, 1:250) and horseradish peroxidase-conjugated goat anti-mouse or anti-rabbit IgG (Jankson ImmunoResearch, 1:1,000 to 1:2,000). The blots were developed with the enhanced chemiluminescence reagents (Amersham Biosciences).

### Microarray Printing, Hybridization and Data Analysis

The details for DNA microarray experiments have been described previously [25]. Briefly, the Pan Rat 10K Oligonucleotide Set (MWG Biotech Inc., High Point, N.C.), containing 6,221 known rat genes, 3,594 rat ESTs, and 169 Arabidopsis negative controls, were printed on epoxy coated slides (CEL Associates, Pearland, Tex.) with an OmniGrid 100 arrayer (GeneMachine, San Carlos, Calif.). After printing, the slides were incubated in 65% humidity overnight at room temperature. The slides were then dried and stored at room temperature until hybridization.

The 2-step microarray hybridization was carried out with the 3DNA 50 Expression kit (Genisphere Inc., Hatfield, Pa.). Prior to hybridization, the slides were washed with 0.2% SDS once and with deionized water for 4 times, and then dried by centrifugation. 5 μg total RNA from each sample were reverse-transcribed into cDNA with a Cy3 (green) or Alexa 647 (Red) specific primer according to the protocol of 3DNA Array 50™ Kit (Genisphere), purified with Microcom YM-30 columns (Millipore, Billerica, Mass.) and dissolved in 1× hybridization buffer (25% formamide, 3×SSC, and 0.1% SDS) at the concentration of 0.3 μg/μl. The EDNA from each sample was paired with a reference cDNA (SuperArray) for hybridization and dye-flip was performed. There were 4 biological replications. The denatured two-color paired cDNA mixture were added to DNA microarray slides and hybridized at 42° C. for 48 hours. After being washed, the slides were re-hybridized with Cy3- and Alexa 647-specific capture reagents at 42° C. for 2 hours and scanned twice (55% PMT and 90% PMT with 90% laser power) with ScanArray Express scanner (PerkinElmer, Boston, Mass.).

The signal intensity for each spot was obtained by Genepix 5.0 (Axon Instruments, Inc. Union City, Calif.). The ratio between each sample and reference cDNA were normalized by LOWESS normalization using the RealSpot software package developed in our laboratory [26]. A quality index (QI) for each spot, based on signal intensity and signal-to-background ratio, was exported from Realspot. The mean QIs were calculated by Excel. Any spots with a mean QI of <1 were filtered. One class SAM statistical test was applied to the remaining genes using a cut-off q-value of <0.05. [31]. The genes that passed the SAM test were clustered by K-means clustering using Cluster and TreeView [32].

### Results

Constructing the pK4-shRNA Vector

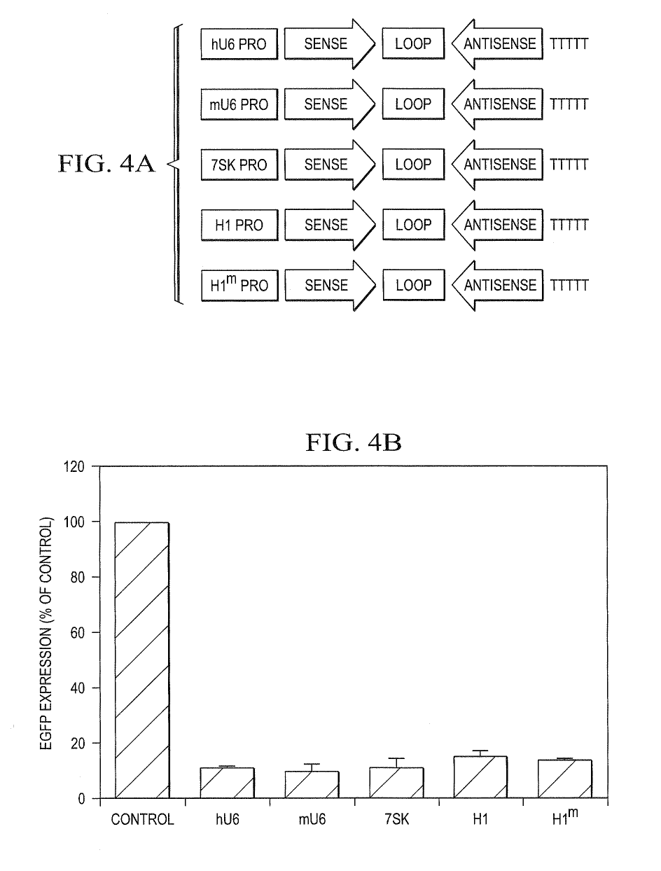

The design of the pK4-shRNA vector features four RNA pol III promoters to direct the intracellular synthesis of four shRNAs. To avoid the problem of DNA recombination in a vector containing multiple identical sequences, four different promoters, mouse U6 (mU6), human U6 (hU6), 7SK, and a mutated H1^{m} (H1^{m}), were selected to construct the pK4-shRNA vector. First, we tested promoter activities by silencing an EGFP reporter gene. We created various constructs, which harbor one promoter and the same shRNA targeted to EGFP at the position of 417-437 by using the pENTR/D-Topo vector (Invitrogen). Each construct was co-transfected with the plasmid pENTR/CMV-EGFP, encoding reporter EGFP and a non-targeted reporter plasmid pDsRed2-C1, encoding DsRed2 protein for normalization. All of the tested promoters had similar EGFP silencing activities in 293A cells (FIG. 4). Similar results were also obtained in rat L2 and mouse NIH-3T3 cell lines (Data not shown). For the convenience of cloning, the Eco31 I restriction site in the H1 promoter was erased by a single point mutation (G→A) at the position of −11. The mutated H1^{m} promoter has the similar silencing efficiency compared to wild type H1 promoters. Therefore, the hU6, mU6, 7SK, and H1^{m} promoters were selected to construct the pK4-shRNA vector. Four well-studied loop sequences (L1: 5′-CTTCCTGTCA-3′ (SEQ ID NO:1), L2: 5′-GGACAGCACAC-3′ (SEQ ID NO:1), L3:5′-TCTCTTGAA-3′ (SEQ ID NO:1) and L4: L4,5′-TTGATATCCG-3′ (SEQ ID NO:1), with the feature of easy PCR amplification were tested in mediating the silencing of EGFP with same promoter and siRNA sequences. We did not find obvious differences in the performance of these loop sequences (Data not shown).

Initially, we constructed the pK4-shRNA vector using 4-step subcloning of different shRNA expression cassettes. Obviously, this procedure was time- and labor-intensive. Since PCR-based amplification is used in almost every aspect of genetic diagnosis, mutation detection and basic research, we attempted to develop a simple strategy to construct the pK4-shRNA vector by the combination of multiple-PCR and one-step cloning. We first generated a bidirectional hU6-H1^{m} promoter in back-to-back form by over-lap PCR (FIG. 3 Step A). Using the hU6-H1^{m} promoter as a template, four sets of primers designed by our in house program, K4-PRIMER, were used to generate the K4-inserts through a four-step PCR amplification (FIG. 3 Step B).

The first PCR was performed with the hU6-H1^{m} template and the P₁-F and P₁-R primers, each annealing to the 3′-end of the hU6 and H1^{m} promoters. The primer-extended PCR products contained (5′) loop 2 (L2), antisense 2 (as2), hU6-H1m, sense 3 (s3), and loop 3 (L3). The second PCR was carried out with the P₂-F and P₂-R primers, which annealed to both ends of the first PCR products based on the complementary sequences of loop 2 and loop 3 at their 3′-ends. A 12-nt linker-1 (Link-1), a stretch of five As (A₅), and a sense 2 (s2), and a 12-nt link-2 (Link-2), a stretch of five Ts (T₅) and an antisense 3 (as3) were added to the upstream and downstream of the second PCR products. Based on the two linker sequences, antisense 1 (as1) with loop 1 (L1) and antisense 4 (as4) with loop 4 (L4) sequences were extended at both ends through the third PCR with the P₃-F and P₃-R primers. The last step of the PCR involved the amplification with the P₄-F and P₄-R primers, each annealing to loop 1 and loop 4. This final PCR product contained hU6 and H1^{m} promoters and four shRNAs with two Eco31 I sites at both ends. The Eco31 I-digested PCR products were cloned into the pre-made vector, pmU6-7SK (FIG. 3 Step C). The pmU6-7SK vector was generated from the pENTR/D-topo vector (Invitrogen) by inserting a mU6-7SK fragment, which containing mU6 and 7SK promoters in a head-to-head orientation. Two Eco31 I sites, in a back-to-back orientation, were engineered at the 3′ end of two promoters. When pmU6-7SK was digested by Eco31 I, CAAA and GAGG overhangs were created on both 5′ ends of the vector, ensuring the ligation to the Eco31 I-digested K4-insert with TTTG and CCTC overhangs at their 5′ end. When we transform the ligation mix into GT116 competent cells, 2 to 3 individual clones for each construct were picked for DNA sequencing. We found that over 50% of the clones had perfect inserts. Additionally, we did not find the problem of DNA rearrangement in over 50 tested plasmids. Unlike regular DNA, K4 inserts contain strong short hairpin structures that may cause difficulty in PCR amplification. To overcome this problem, several DNA polymerases have been tested at different conditions. We found only Advance 2 Taq polymerase (BD Science) at the optimized condition can produce consistent amplification in every PCR reaction.

Evaluating the Silencing Efficiency of pK4-shRNA Compared to Individual shRNA Vector

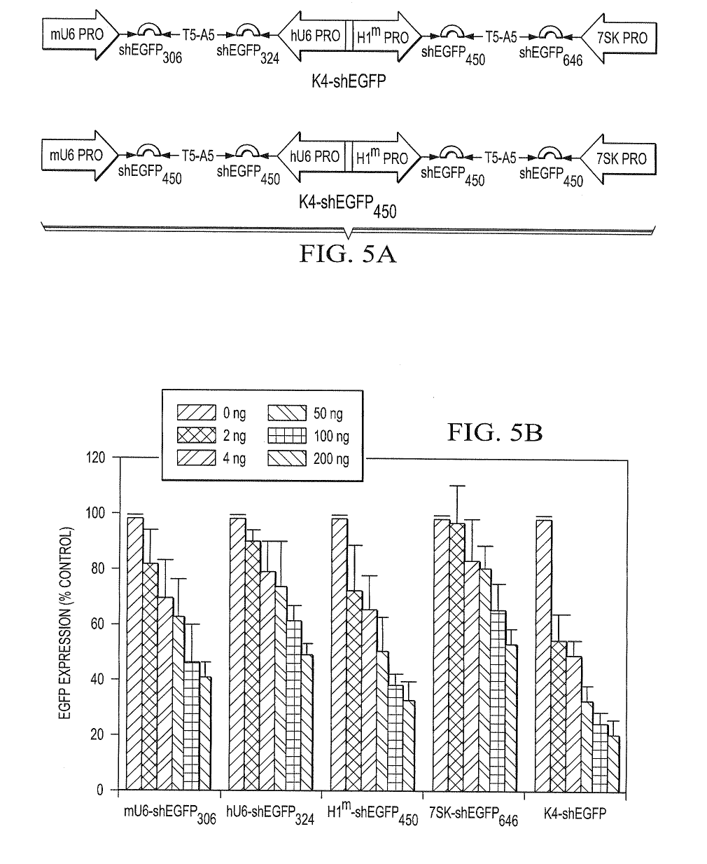

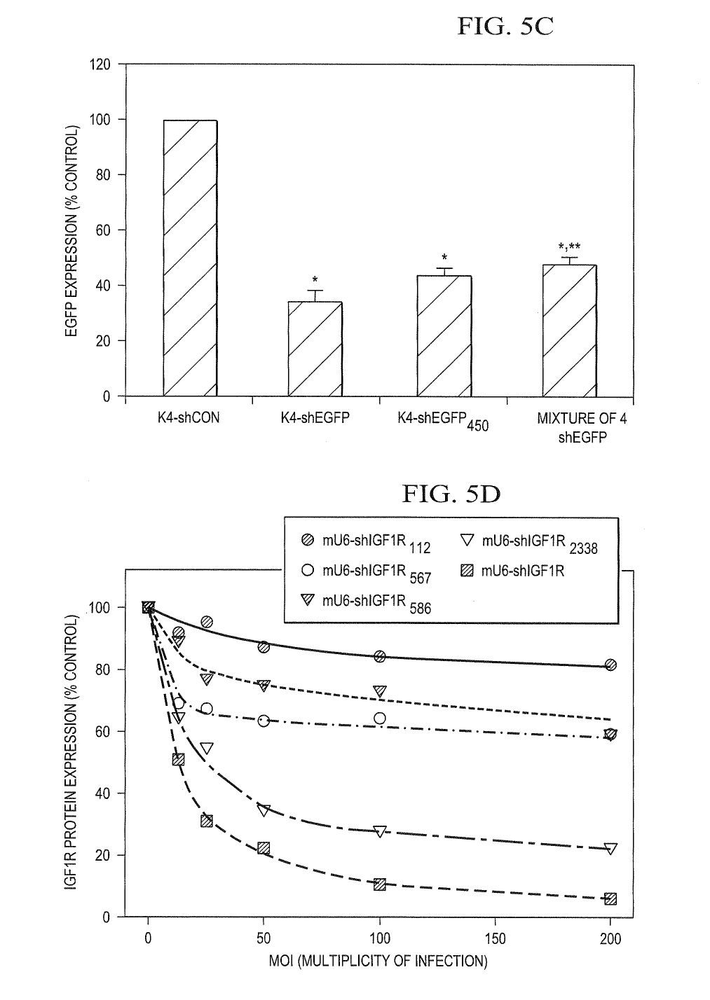

To evaluate the effectiveness of pK4-shRNA, we selected four siRNAs with relatively weak activities against EGFP at the position of 306-326, 324-344, 450-470, and 646-666 for constructing pK4-shEGFP (FIG. 5a). For comparison, we also made four single shRNA vectors containing one siRNA and the corresponding promoter: pmU6-shEGFP₃₀₆, phU6-shEGFP₃₂₄, pH1^{m}-shEGFP₄₅₀ and p7SK-shEGFP₆₄₆. EGFP expression was only reduced by 8-27% and 46-65% in the 293A cells treated with 2 and 200 ng of individual shRNAs, respectively; however, the inhibition of EGFP by the pK4-shEGFP vector was increased to 44% and 80% of EGFP expression at the same dose (FIG. 5b). Similar experiments using the rat L2 cell line yielded the same results (data not shown). These experiments show that simultaneous expression of multiple shRNAs against different regions of a mRNA effectively improves the efficiency of knockdown over a single shRNA construct, which is a finding consistent with previous reports [3,20,23,27].

We further compared the differences in silencing gene between one vector harboring 4 shRNAs (K4-shEGFP) and a mixture of 4 individual vectors harboring one shRNA (Mixture of 4 shEGFP). The 293A cells were transfected with an equal amount of the K-4-shEGFP vector (20 ng) and the mixture of 4 single shRNA vector (total 20 ng and 5 ng each). As shown in FIG. 5c, the vector expressing 4 shRNAs has a higher silencing efficiency (65±6.3%) than a mixture of 4 vectors expressing a single shRNA (49±7.4%) (FIG. 5c).

Logically, it is easy to accept that the multiple shRNAs could achieve better knockdown than a single shRNA. The reason may be due to additive or synergistic effects of multiple shRNAs. To address this point, the best EGFP siRNA sequence at the position of 450-470 in pK4-shEGFP vector was selected to build a new vector, pK4-shEGFP₄₅₀, in which four copies of shEGFP450 were transcribed under the control of different promoters (FIG. 5a). When comparing the silencing ability, we found that pK4-shEGFP exhibited a higher inhibition of EGFP expression (65±6.3%) than pK4-shEGFP₄₅₀ (54±4.8%) (FIG. 5c), even though shEGFP₄₅₀ is the most effective sequence among the four siRNAs. The result indicates that the siRNAs binding to different positions of the target mRNA may have a synergic effect on gene silencing.

We next tested the effectiveness of endogenous gene silencing with the pK4-shRNA system. As adenoviruses can infect a wide range of cell lines and primary cells, we use the pK4-shRNA adenoviral vector for this purpose. Two plasma membrane proteins, insulin-like growth factor receptor 1 (IGF1R) and SNAP-23, were tested first. IGF1R is a key regulator of cell growth and development [28], whereas SNAP-23 plays a critical role in intracellular trafficking [29]. The expression of IGF1R protein in RLE-6NT cells, a rat lung type II cell line, was only marginally affected by three of the four single shRNAs, while another one, shIGF1R₂₂₃₈ led to a reduction of 70% at the 100 MOI dose. However, at the same dose of 100 MOI, pK4-shIGF1R increased the silencing efficiency to ˜93% (FIG. 5d). Similarly, single shRNAs targeted to SNAP-23 reduced SNAP-23 mRNA ˜60 to 80% in RLE-6NT cells at the 100 MOI viral dose, while the simultaneous expression of all 4 siRNAs within pK4-shSNAP-23 resulted in a suppression of >97% (FIG. 5e), indicating that gene knockdown efficiency of a single shRNA, except for certain single shRNA vectors that can already achieve near-complete knockdown, can be significantly improved by the application of our K4-shRNA design. To achieve ˜90% inhibition of SNAP-23 by the pK4-shSNAP-23 viral vector, the dose of virus can be decreased to 25 MOI, significantly reducing the amount of virus required to achieve equivalent silencing. This would alleviate the pro-inflammatory effect of adenovirus as well as off-target effects.

It has been demonstrated by several groups that a multiple shRNA approach is better than single shRNA [12-23,30]. Our initial purpose of developing a pK4-shRNA vector was to see whether this strategy would circumvent the need of screening individual effective siRNAs. Therefore, we constructed 16 pK4-shRNA vectors targeted to 12 different endogenous genes and tested their silencing abilities in cell lines and/or primary lung type II cells. As measured by real-time PCR or Western blot, we found that all of those vectors can achieve over 70% of knockdown and 13 of the K4-shRNA vectors can produce more than 85% inhibition (Table 1). These results indicate that our K4-shRNA system holds significant promise for eliminating the initial siRNA screening step given that 25% of the selected target siRNA sequences are functional with more than 75% knockdown efficiency.

TABLE 1
Summary of pK4-shRNA vectors and their silencing efficiencies

| | | | | mRNA or |
|---|---|---|---|---|
| | | Selected four siRNA | Infected | protein |
| Name of | Target | sequences (and | cells or | reduction |
| construct | gene | their positions) | tissue | (%) |
| pK4- | IGF1R | 5′-GACATCCGCAACGACTATCA-3′ | Rat | Protein |
| shIGF1R | | (112-131) | primary | ~95 |
| | | (SEQ ID NO: 41) | lung type | |
| | | 5′-GCCCATGTGTGAGAAGACCA-3′ | II cells | |
| | | (567-586) | | |
| | | (SEQ ID NO: 42) | | |
| | | 5′-ACCATCAACAATGAGTACAA-3′ | | |
| | | (586-605) | | |
| | | (SEQ ID NO: 43) | | |
| | | 5′-GAGAGCAGAGTGGATAACAA-3′ | | |
| | | (2338-2357) | | |
| | | (SEQ ID NO: 44) | | |
| pK4- | IGF1R | 5′-CTGTATCTCAGTGGATCTTCA-3′ | Rat | Protein |
| shIGF1Rnc | | (4231-4251)* | primary | ~92 |
| | | (SEQ ID NO: 45) | lung type | |
| | | 5′-GAGAATTGAGTCTCCTCATTC-3′ | II cells | |
| | | (4418-4438)* | | |
| | | (SEQ ID NO: 46) | | |
| | | 5′-CTGCCTGAGCACCATAGGTCT-3′ | | |
| | | (4606-4626)* | | |
| | | (SEQ ID NO: 47) | | |
| | | 5′-AACCTTAATGACAGCTCTTAAT-3′ | | |
| | | (4381-4402)* | | |
| | | (SEQ ID NO: 48) | | |
| pK4-Smad 4 | Smad 4 | 5′-GGTGGAGAGAGTGAGACATT-3′ | Rat | Protein |
| | | (85-104) | primary | ~98 |
| | | (SEQ ID NO: 49) | lung type | |
| | | 5′-GCGTCTGTGTGAACCCATATC-3′ | II cells | |
| | | (374-394) | | |
| | | (SEQ ID NO: 50) | | |
| | | 5′-GGAATTGATCTCTCTGGATTA-3′ | | |
| | | (418-438) | | |
| | | (SEQ ID NO: 51) | | |
| | | 5′-GGAGTGCAGTTGGAGTGTAAA-3′ | | |
| | | (1156-1176) | | |
| | | (SEQ ID NO: 52) | | |
| pK4-Smad | Smad 4 | 5′-GTCTTCACTGGTTGTTATGTA-3′ | Rat | Protein |
| 4nc | | (1898-1918)* | primary | ~96 |
| | | (SEQ ID NO: 53) | lung type | |
| | | 5′-GTTAAGTCACCTGTTACTTAG-3′ | II cells | |
| | | (2053-2073)* | | |
| | | (SEQ ID NO: 54) | | |
| | | 5′-GCAGAGTTGCTCTGCCTGATG-3′ | | |
| | | (2498-2518)* | | |
| | | (SEQ ID NO: 55) | | |
| | | 5′-CTAATCTGTGTGCATATTGAC-3′ | | |
| | | (2256-2276)* | | |
| | | (SEQ ID NO: 56) | | |
| pK4-shAIIa | Annexin A2 | 5′-TTATACACTCGGTTAATCTCC-3′ | Rat | mRNA |
| | | (423-443) | primary | ~95 |
| | | (SEQ ID NO: 57) | lung type | Protein |
| | | 5′-GACATCATCTCTGACACATCT-3′ | II cells | ~95 |
| | | (472-492) | | |
| | | (SEQ ID NO: 58) | | |
| | | 5′-ACACCAACTTCGACGCTGAGA-3′ | | |
| | | (89-109) | | |
| | | (SEQ ID NO: 59) | | |
| | | 5′-ATTGTCAACATTCTGACTAA-3′ | | |
| | | (166-185) | | |
| | | (SEQ ID NO: 60) | | |
| pK4-shAIIb | Annexin A2 | 5′-AATGCACAGAGGCAGGACATT-3′ | Rat | mRNA |
| | | (193-213) | primary | ~96 |
| | | (SEQ ID NO: 61) | lung type | Protein |
| | | 5′-GTGCCTATGGGTCGGTCAAAC-3′ | II cells | ~94 |
| | | (65-85) | | |
| | | (SEQ ID NO: 62) | | |
| | | 5′-AGAGCTACAGTCCTTATGACA-3′ | | |
| | | (698-718) | | |
| | | (SEQ ID NO: 63) | | |
| | | 5′-ACATTGAAACAGCAATCAAGA-3′ | | |
| | | (122-142) | | |
| | | (SEQ ID NO: 64) | | |
| pK4-shPTN | Pleiotrophin | 5′-GCACTGGTGCCGAGTGCAAAC-3′ | Fetal rat | Protein |
| | | (212-232) | lung | ~91 |
| | | (SEQ ID NO: 65) | fibroblasts | mRNA |
| | | 5′-GATCCCTTGCAACTGGAAGAA-3′ | | ~97 |
| | | (258-278) | | |
| | | (SEQ ID NO: 66) | | |
| | | 5′-CCATGAAGACTCAGAGATGTA-3′ | | |
| | | (236-256) | | |
| | | (SEQ ID NO: 67) | | |
| | | 5′-GCACAATGCCGACTGTCAGAA-3′ | | |
| | | (378-398) | | |
| | | (SEQ ID NO: 68) | | |
| pK4- | Pleiotrophin | 5′-ATTTATACCTACTGTAGGCTT-3′ | Fetal rat | Protein |
| shPTNnc | | (570-590)* | lung | ~91 |
| | | (SEQ ID NO: 69) | fibroblasts | mRNA |
| | | 5′-GCAGGATCAGTTAACTATTAC-3′ | | ~90 |
| | | (549-569)* | | |
| | | (SEQ ID NO: 70) | | |
| | | 5′-CTGTAGCTTAAGTACATGATA-3′ | | |
| | | (607-627)* | | |
| | | (SEQ ID NO: 71) | | |
| | | 5′-ACTACTTCCCTTATTAGATAG-3′ | | |
| | | (909-929)* | | |
| | | (SEQ ID NO: 72) | | |
| pK4- | Beta- | 5′-GGACCAGGTGGTCGTTAATAA-3′ | Rat fetal | mRNA |
| shCatenin | catenin | (489-509) | lung type | ~90 |
| | | (SEQ ID NO: 73) | II cells | |
| | | 5′-GTGGATTCCGTACTGTTCTAC-3′ | | |
| | | (742-762) | | |
| | | (SEQ ID NO: 74) | | |
| | | 5′-GAATGCCGTTCGCCTTCATTA-3′ | | |
| | | (1446-1466) | | |
| | | (SEQ ID NO: 75) | | |
| | | 5′-ACTGTTGGATTGATCCGAAAC-3′ | | |
| | | (1528-1548) | | |
| | | (SEQ ID NO: 76) | | |
| pK4- | SNAP-23 | 5′-GGATGATCTATCACCAGAAGA-3′ | RLE-6NT | mRNA |
| shSNAP-23 | | (3-23) | cells | ~97 |
| | | (SEQ ID NO: 77) | Rat | Protein |
| | | 5′-GAAGGCATGGACCAAATAA-3′ | primary | ~94 |
| | | (169-187) | lung type | |
| | | (SEQ ID NO: 78) | II cells | |
| | | 5′-CTAATGATGCCAGAGAAGA-3′ | | |
| | | (428-446) | | |
| | | (SEQ ID NO: 79) | | |
| | | 5′-CAAGAATCGCATTGACATTG-3′ | | |
| | | (579-598) | | |
| | | (SEQ ID NO: 80) | | |
| pK4- | Rab 14 | 5′-CACCGTACAACTACTCTTACA-3′ | Rat | mRNA |
| shRab14 | | (28-48) | primary | ~98 |
| | | (SEQ ID NO: 81) | lung type | |
| | | 5′-GGCTGATTGTCCTCACACAAT-3′ | II cells | |
| | | (84-103) | | |
| | | (SEQ ID NO: 82) | | |
| | | 5′-GAATTTGGTACAAGAATAATT-3′ | | |
| | | (199-217) | | |
| | | (SEQ ID NO: 83) | | |
| | | 5′-GTTACACGGAGCTACTATAGA-3′ | | |
| | | (384-405) | | |
| | | (SEQ ID NO: 84) | | |
| pK4-shp11 | p11 | 5′-GAAACCATGATGCTTACATTT-3′ | Rat | mRNA |
| | | (28-48) | primary | ~95 |
| | | (SEQ ID NO: 85) | lung type | |
| | | 5′-GGAGGACCTGAGAGTGCTCA-3′ | II cells | |
| | | (84-103) | | |
| | | (SEQ ID NO: 86) | | |
| | | 5′-GTGGGCTTCCAGAGCTTTCTA-3′ | | |
| | | (199-217) | | |
| | | (SEQ ID NO: 87) | | |
| | | 5′-CCTTAGGAAATGTGCAAATAA-3′ | | |
| | | (384-405)* | | |
| | | (SEQ ID NO: 88) | | |
| pK4- | Duox2 | 5′-GCTACGACGGCTGGTTTAATA-3′ | Rat fetal | mRNA |
| shDuox2 | | (110-130) | lung type | ~87 |
| | | (SEQ ID NO: 89) | II cells | |
| | | 5′-GAACATTGCTCTATACCAATG-3′ | | |
| | | (882-902) | | |
| | | (SEQ ID NO: 90) | | |
| | | 5′-ACGCAAGATGCTACTAAAGAA-3′ | | |
| | | (1878-1898) | | |
| | | (SEQ ID NO: 91) | | |
| | | 5′-CCTCATGACATAGCAAGTTAT-3′ | | |
| | | (4696-4716)* | | |
| | | (SEQ ID NO: 92) | | |
| pK4- | Bglap | 5′-CAGTAAGGTGGTGAATAGACT-3′ | Rat fetal | mRNA |
| shBglap | | (120-140) | lung type | ~75 |
| | | (SEQ ID NO: 93) | II cells | |
| | | 5′-CGCTACCTCAACAATGGACTT-3′ | | |
| | | (145-165) | | |
| | | (SEQ ID NO: 94) | | |
| | | 5′-GACGAGCTAGCGGACCACATT-3′ | | |
| | | (235-255) | | |
| | | (SEQ ID NO: 95) | | |
| | | 5′-CATCTATGGCACCACCGTTTA-3′ | | |
| | | (279-299) | | |
| | | (SEQ ID NO: 96) | | |
| pK4-shNelf | Nelf | 5′-ATTGAGCTAGCAGTGGTGAAA-3′ | Rat fetal | mRNA |
| | | (355-375) | lung type | ~73 |
| | | (SEQ ID NO: 97) | II cells | |
| | | 5′-AGGATGTATAGTGTTGATGGA-3′ | | |
| | | (607-627) | | |
| | | (SEQ ID NO: 98) | | |
| | | 5′-CCACAACTATGCAAGCCATCT-3′ | | |
| | | (695-715) | | |
| | | (SEQ ID NO: 99) | | |
| | | 5′-GAATGATTCCGCGTCTGTAAT-3′ | | |
| | | (759-779) | | |
| | | (SEQ ID NO: 100) | | |
| pK4-shDlk1 | Dlk1 | 5′-ACCACATGCTTCGCAAGAAGA-3′ | Rat fetal | mRNA |
| | | (1154-1174) | lung type | ~71 |
| | | (SEQ ID NO: 101) | II cells | |
| | | 5′-GGAAGGCTGGGACGGGAAATT-3′ | | |
| | | (366-386) | | |
| | | (SEQ ID NO: 102) | | |
| | | 5′-GGAGGCTGGTGATGAGGATAT-3′ | | |
| | | (1263-1283) | | |
| | | (SEQ ID NO: 103) | | |
| | | 5′-ATCTAGTGAACGCTACGCTTA-3′ | | |
| | | (1397-1417) | | |
| | | (SEQ ID NO: 104) | | |

*indicates that the sequences were selected from the 3′-noncoding region.

### Simultaneous Knockdown of Four Different Genes

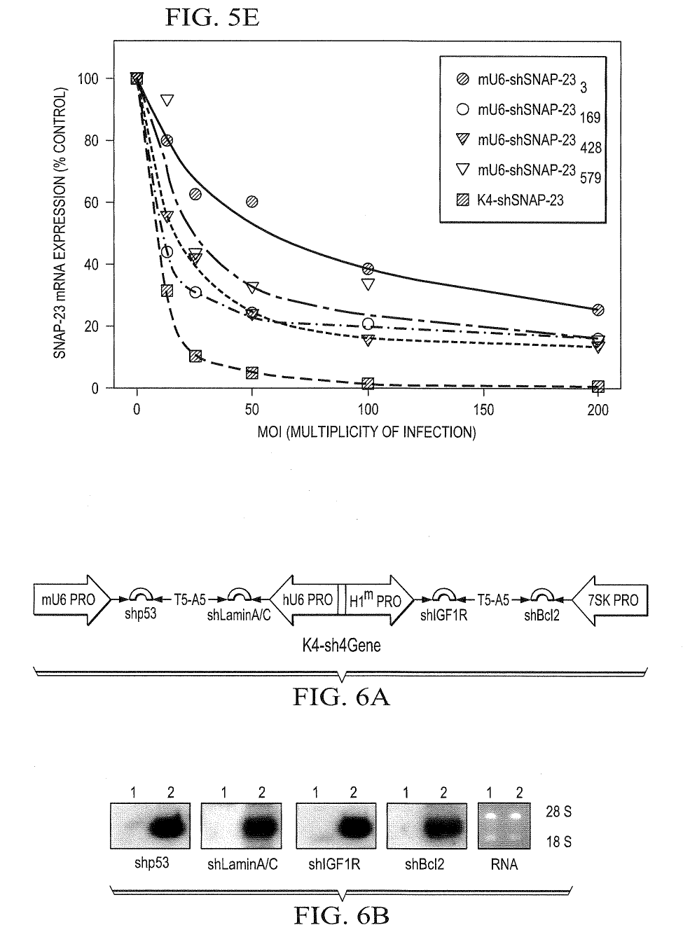

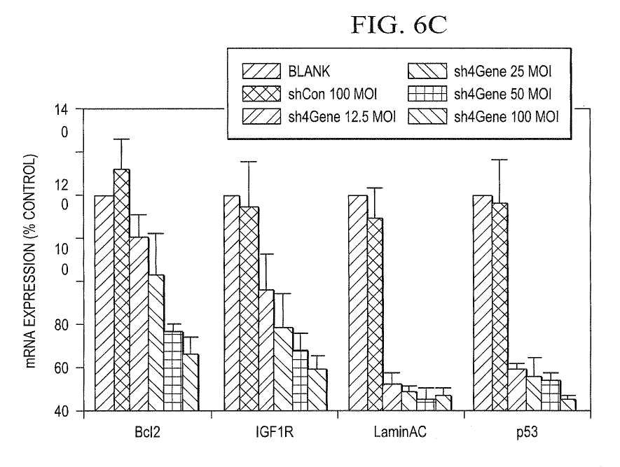

It has been demonstrated that double or triple shRNA vectors can knockdown different target genes simultaneously without significant competitive inhibition by the inclusion of multiple shRNAs [15,17,21,23]. To test whether the K4-shRNA design can knockdown four different proteins and also whether there is a potential promoter conflict between each Pol-III promoter, we selected four different human genes, Lanin A/C, p53, IGF1R and Bcl2. According to the reported siRNA sequences for each target [3, 31-33], we constructed a new vector, K4-sh4Gene, in which the shRNA transcripts for p53, Lamin A/C, IGF1R and Bcl2 were controlled by mU6, hU6, H1^{m} and 7SK promoters, respectively (FIG. 6a). Northern blot analysis revealed that the four shRNAs were expressed at similar sizes and abundance in K4-sh4Gene infected-A549 cells (FIG. 6b), indicating that no apparent competition exists between multiple pol III promoters in close proximity. The mRNA levels of all the target genes were reduced to various extents. When A549 cells infected by K4-sh4Gene adenovirus at 100 MOI, simultaneous inhibitions of p53, IGF1R, Lamin A/C and Bcl2 were about 95.2±1.6%, 81.2±6.5%, 93.3±2.3% and 73.1±7.5%, respectively (FIG. 6c), comparable to the reported silencing efficiencies of p53 [3], 95%; IGF1R [31], 80-95%; Lamin A/C [32], >90%; and Bcl2 [33], 82%. Our results indicate that it is feasible to introduce four shRNAs to silence different genes simultaneously with little or no reduction in efficacy.

Specificity of the pK4-shRNA Vector

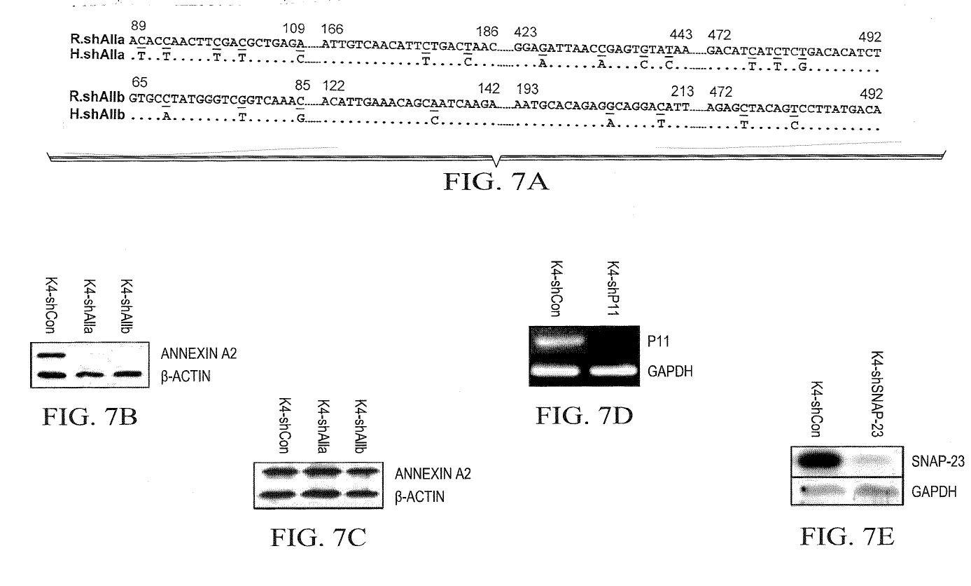

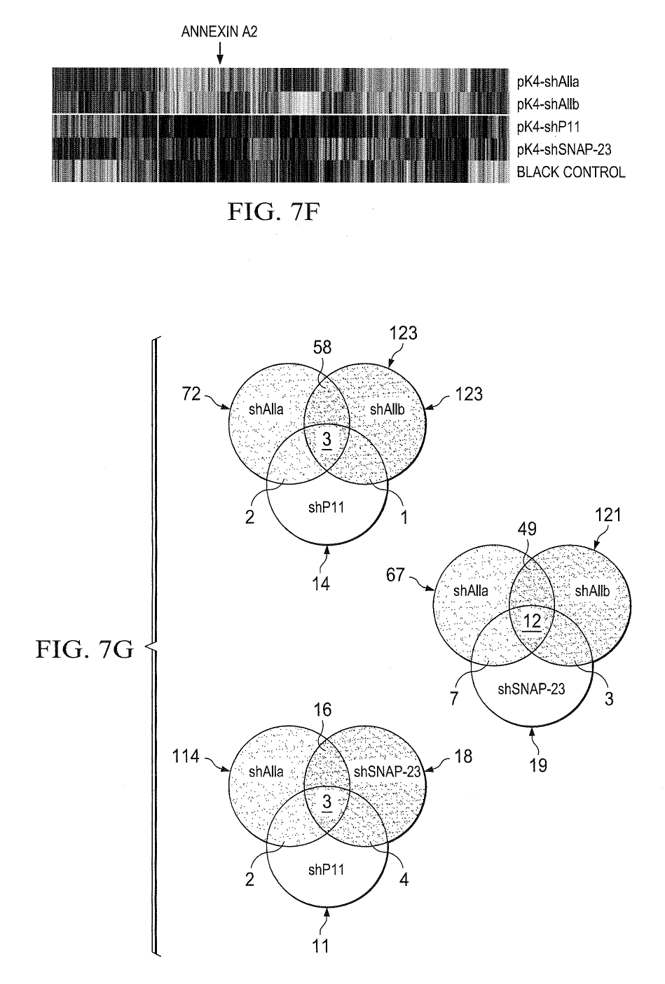

As a useful shRNA expression vector in gene knockdown application, especially in future gene therapy, the specificity of inhibition by shRNA to the target is an important consideration. Recent reports suggest that off-target effects can occur from siRNAs, at the level of both mRNA and protein [34]. Therefore, careful attention has been paid for an evaluation of pK4-shRNA-based gene silencing. First, we examined the inhibition of annexin A2 in different species. Annexin A2 is a cytosolic Ca²⁺-dependent phospholipid-binding protein that plays an important role in membrane fusion during exocytosis [35]. Two sets of four siRNAs (pK4-shAIIa and pK4-shAIIb) were selected from the coding region of rat annexin A2 (FIG. 7a). Compared to the control vector, over 95% of annexin A2 protein was depleted from primary rat alveolar type II cells transduced with 50 MOI pK4-shAIIa or pK4-shAIIb adenovirus (FIG. 7b). There were 1-5 base mismatches in the regions of either rat or human annexin A2, in which the four siRNAs of pK4-shAIIa or pK4-shAIIb were targeted (FIG. 7a). When we infected human A549 cells with pK4-shAIIa or pK4-shAIIb adenovirus targeted to rat sequences, both vectors had little effect on human annexin A2 expression (FIG. 7c). The result indicates that the silencing of rat annexin A2 by pK4-shRNA is sequence-specific.

Because examining only one or a few genes is not enough for testing RNAi specificity, we performed gene expression profiling analysis to detect the potential off-target effects of pK4-shRNA at an unbiased, genomic scale using DNA microarray containing 10,000 genes. We reasoned that if the pK4-shRNAs elicited a target-specific response, the pK4-shRNA vectors targeted to annexin A2, regardless of the target regions, should induce similar changes in the gene expression profiles compared to the pK4-shRNA targeted to other genes. We chose two pK4-shRNAs targeted to different regions of rat annexin A2 (pK4-shAIIa and pK4-shAIIb) (FIG. 7a), which have similar silencing efficiency (FIG. 7b). We also constructed additional two vectors, pK4-shP11 and pK4-shSANP-23 targeting to P11 and SNAP-23. P11 (or S100A10), a member of the S100 family of Ca²⁺-binding proteins, is found in most cells. It binds to annexin A2 to form a heterotetrameric complex, (S100A10)₂ (annexin A2)₂. SNAP-23 is a 23 kDa synaptosome-associated protein that highly expressed in alveolar epithelial type II cells. SNAP-23 is involved in the process of membrane fusion in the exocytosis of lamellar bodies in type II cells. Because both P11 and SNAP-23 are structurally different, but functionally related to annexin A2, we selected them as controls for the evaluation of off-target effects of pK4-shAII. pK4-shP11 and pK4-shSNAP-23 reduced the expression of p11 and SNAP-23 in primary type II cells by 95% and 94%, respectively (FIGS. 7d and 7e).

The DNA microarray was then used to determine the changes in global gene expression in untreated type II cells (blank control) and the type II cells infected with pK4-shAIIa, pK4-shAIIb, pK4-p11, pK4-SNAP-23 or the control vector, pK4-shCon adenovirus, Each of the 6 samples was co-hybridized with a reference RNA (Ref) from SuperArray using a reference design as follows: pK4-shAIIa/Ref, pK4-shAIIb/Ref, pK4-shP11/Ref, pK4-shSNAP-23/Ref, pK4-shCon/Ref and blank control/Ref. There were total 48 hybridizations with 4 biological replications and dye flipping. After filtering the bad and weak spots, the remaining good spots were analyzed by statistical SAM test. The genes that passed the SAM test were subjected to cluster analysis. As shown in FIG. 5f, the gene expression signatures generated by the pK4-shRNAs against the same target of annexin A2 (K4-shAIIa and K4-shAIIb) were more similar than the pK4shRNAs targeted to the different genes (K4-shP11 and K4-shSNAP-23). The common genes due to the treatment of K4-shRNAs against annexin A2, P11 and SNAP-23 were presented by Venn diagrams (FIG. 5g). It is clear that annexin A2 only decreased when treated with the relevant shRNAs. We found 61 commonly changed genes between two pK4-shRNA vectors targeted to the same gene of annexin A2, K4-shAIIa and K4shAIIb; however, 4-19 genes were common between any pairs of pK4-shRNA vectors targeted to different genes, annexin A2, p11 and SNAP-23. The observed quantitative and qualitative similarities between different pK4-shRNAs against the same gene were higher than pK4-shRNAs against different genes, suggesting that the knockdown signatures are unique to each gene.

### 2. Discussion

Vector-based RNAi has become a popular approach for analyzing gene function in mammalian cells. Recently, several laboratories have reported that effective knockdown can be achieved by multiple shRNAs in a single vector. Moreover, the expression of up to three different proteins can be depressed simultaneously [12,14-23,30]. For the construction of multiple shRNAs vector, the most common design is achieved by several steps of subcloning of different shRNA expression cassettes [13,15,17,23]. Obviously, this method is costly and time-consuming.

Here, we describe a new strategy of cloning a single plasmid expressing four shRNAs. The advantages of our method are as follows: First, it increases the vector stability, decreases cost and saves time. The common methods of constructing multiple shRNA vectors were achieved by cloning different expression cassettes with the same promoter in tandem orientation [15,17]. As multiple repeats of identical sequences in a single vector poses a severe problem for DNA recombination which may result in deletion of one or multiple repeats and the intervening sequence in E. coli, it would take a lot of effort to screen the colony without DNA rearrangement. Additionally, transfection of such a plasmid into mammalian cells may still have the risk of gene rearrangements. Increasing the vector amount may be able to minimize the net effect of this phenomenon; however, other undesirable side effects may be induced by the concentrated DNA or virus-mediated shRNA in transfected cells, not to mention that the cost would be increased. Another option to express multiple shRNAs can be obtained from polycistronic transcripts under the control of a pol 11 promoter, such as the CMV or Ubc promoters [20,21]. The polycistronic transcripts were designed to mimic branched microRNA precursors. However, such RNA structures are complex and difficult in making the construction. To avoid recombination as well as reduce cost, we selected four different promoters for shRNA expression in a single vector. All of the promoters used have been well studied and used in different mammalian cells, although their expression efficiencies have slight differences in some cell types [36]. To save time and cost during constructing the vector, the annealing sequences in each primer were optimized for a four-step PCR amplification. The size of all primers was less than 50-mer, making it is possible to be synthesized at the 0.05 micromole scale without a PAGE purification step. We also tested a two-step PCR with four longer primers, however, we found that it was costly in primer synthesis and purification and also increased the possibility of shRNA mutations. The method described here is a one-step cloning process that dramatically saves time in vector construction. We also found that the mutation rate in the shRNA sequences was considerably reduced by our method. Based on the sequencing of 16 constructs, we found at least one clone out of 2 had the correct sequences in all 4 shRNA sequences. Second, the pENTR-derived pK4-shRNA vector can be directly switched to an adenoviral or lentiviral system by gateway techniques. Therefore, it can be applied to primary cell and organ culture. Third, the 4 shRNA system makes it possible to reduce or eliminate screening of effective siRNA sequences. Of the 16 constructs tested, we found that all of the K4-shRNA constructs could knockdown the target genes by over 70% and 13 constructs could induce over 85% inhibition. Fourth, the combined different shRNAs resulted in effective and simultaneous depression of four targets, while their individual activity was maintained. Although the silencing of two or three genes by a single vector was reported [12,13,15,17,20,21,23], our design can silence up to four target proteins, thus providing a more efficient tool for RNAi therapy. Recently, several groups demonstrated that, when a multiple shRNA strategy was used to target different conserved regions of HIV-1, the magnitude of inhibition was dramatically increased, approaching a complete inhibition. Also, the chance of escape was reduced [15,22]. Since pK4-shRNA is capable of expressing four different shRNAs, we believe that this system would be more useful to achieve longer inhibition of viruses.

In summary, we present a simple, quick and cost-effective method to construct multiple shRNAs expression vectors driven by different pol III promoters. With this approach, silencing efficiencies of single shRNA constructs can be significantly improved. The method also features the silencing of four different genes simultaneously, further extending the application spectrum of RNAi, both in functional studies and therapeutic strategies.

In view of the above, it will be seen that the objective of the invention is achieved and other advantageous results attained. As various changes could be made without departing from the scope of the invention, it is intended that all matter contained in the above description or shown in the accompanying drawings shall be interpreted as illustrative and not in a limiting sense.

While the invention has been described with a certain degree of particularity, it is understood that the invention is not limited to the embodiment(s) set for herein for purposes of exemplification, but is to be limited only by the scope of the attached claim or claims, including the full range of equivalency to which each element thereof is entitled.

### REFERENCES

1. Fire A, Xu S, Montgomery M K, Kostas S A, Driver S E, Mello C C: Potent and specific genetic interference by double-stranded RNA in Caenorhabditis elegans. Nature 1998, 391:806-811.
2. Kurreck J: siRNA Efficiency: Structure or Sequence-That Is the Question. J Biomed Biotechnol 2006, 2006:83757.
3. Brummelkamp T R, Bernards R, Agami R: A system for stable expression of short interfering RNAs in mammalian cells. Science 2002, 296:550-553.
4. Sui G, Soohoo C, Affar eB, Gay F, Shi Y, Forrester W C: A DNA vector-based RNAi technology to suppress gene expression in mammalian cells. Proc Natl Acad Sci USA 2002, 99:5515-5520.
5. Singer O, Yanai A, Verma I M: Silence of the genes. Proc Natl Acad Sci USA 2004, 101:5313-5314.
6. Kim D H, Behlke M A, Rose S D, Chang M S, Choi S, Rossi J J: Synthetic dsRNA Dicer substrates enhance RNAi potency and efficacy. Nat Biotechnol 2005, 23:222-226.
7. Myers J W, Jones J T, Meyer T, Ferrell J E, Jr.: Recombinant Dicer efficiently converts large dsRNAs into siRNAs suitable for gene silencing. Nat Biotechnol 2003, 21:324-328.
8. Yang D, Buchholz F, Huang Z, Goga A, Chen C Y, Brodsky F M, Bishop J M: Short RNA duplexes produced by hydrolysis with Escherichia coli RNase III mediate effective RNA interference in mammalian cells. Proc Natl Acad Sci USA 2002, 99:9942-9947.
9. Luo B, Heard A D, Lodish H F: Small interfering RNA production by enzymatic engineering of DNA (SPEED). Proc Natl Acad Sci USA 2004, 101:5494-5499.
10. Shirane D, Sugao K, Namiki S, Tanabe M, Jino M, Hirose K: Enzymatic production of RNAi libraries from cDNAs. Nat Genet. 2004, 36:190-196.
11. Mittal V: Improving the efficiency of RNA interference in mammals. Nat Rev Genet. 2004, 5:355-365.
12. Chung K H, Hart C C, Al-Bassam S, Avery A, Taylor J, Patel P D, Vojtek A B, Turner D L: Polycistronic RNA polymerase II expression vectors for RNA interference based on BIC/miR-155. Nucleic Acids Res 2006, 34:e53.
13. Gonzalez S, Castanotto D, Li H, Olivares S, Jensen M C, Forman S J, Rossi J J, Cooper L J: Amplification of RNAi—targeting HLA mRNAs. Mol Ther 2005, 11:811-818.
14. Henry S D, van der W P, Metselaar H J, Tilanus H W, Scholte B J, van der Laan LJ: Simultaneous targeting of HCV replication and viral binding with a single lentiviral vector containing multiple RNA interference expression cassettes. Mol Ther 2006, 14:485-493.
15. Ter B O, Konstantinova P, Ceylan M, Berkhout B: Silencing of HIV-1 with RNA Interference: a Multiple shRNA Approach. Mol Ther 2006, 14:883-892.
16. Hung C F, Cheng T L, Wu R H, Teng C F, Chang W T: A novel bidirectional expression system for simultaneous expression of both the protein-coding genes and short hairpin RNAs in mammalian cells. Biochem Biophys Res Commun 2006, 339:1035-1042.
17. Jazag A, Kanai F, Ijichi H, Tateishi K, Ikenoue T, Tanaka Y, Ohta M, Imamura J, Guleng B, Asaoka Y et al.: Single small-interfering RNA expression vector for silencing multiple transforming growth factor-beta pathway components. Nucleic Acids Res 2005, 33:e131.
18. Miyagishi M, Sumimoto H, Miyoshi H, Kawakami Y, Taira K: Optimization of an siRNA-expression system with an improved hairpin and its significant suppressive effects in mammalian cells. J Gene Med 2004, 6:715-723.
19. Schuck S, Manninen A, Honsho M, Fullekrug J, Simons K: Generation of single and double knockdowns in polarized epithelial cells by retrovirus-mediated RNA interference. Proc Natl Acad Sci USA 2004, 101:4912-4917.
20. Sun D, Melegari M, Sridhar S, Rogler C E, Zhu L: Multi-miRNA hairpin method that improves gene knockdown efficiency and provides linked multi-gene knockdown. Biotechniques 2006, 41:59-63.
21. Zhou H, Xia X G, Xu Z: An RNA polymerase II construct synthesizes short-hairpin RNA with a quantitative indicator and mediates highly efficient RNAi. Nucleic Acids Res 2005, 33:e62.
22. Anderson J, Akkina R: HIV-1 resistance conferred by siRNA cosuppression of CXCR4 and CCR5 coreceptors by a bispecific lentiviral vector. AIDS Res Ther 2005, 2:1.
23. Schubert S, Grunert H P, Zeichhardt H, Werk D, Erdmann V A, Kurreck J: Maintaining inhibition: siRNA double expression vectors against coxsackieviral RNAs. J Mol Biol 2005, 346:457-465.
24. Gou D, Narasaraju T, Chintagari N, Jin N, Wang P, Liu L: Gene silencing in alveolar type II cells using cell-specific promoter in vitro and in vivo. Nucleic Acids Res 2004, 32:e134.
25. Chen Z, Chen J W, Weng T, Jin N, Liu L: Identification of rat lung-prominent genes by a parallel DNA microarray hybridization. BMC Genomics 2006, in press.
26. Chen Z, Liu L: RealSpot: Software validating results from DNA microarray data analysis with spot images. Physiol Genomics 2005, 21:284-291.
27. Gou D, Jin N, Liu L: Gene silencing in mammalian cells by PCR-based short hairpin RNA. FEBS Lett 2003, 548:113-118.
28. Baker J, Liu J P, Robertson E J, Efstratiadis A: Role of insulin-like growth factors in embryonic and postnatal growth. Cell 1993, 75:73-82.
29. Kawanishi M, Tamori Y, Okazawa H, Araki S, Shinoda H, Kasuga M: Role of SNAP23 in insulin-induced translocation of GLUT4 in 3T3-L1 adipocytes. Mediation of complex formation between syntaxin4 and VAMP2. J Biol Chem 2000, 275:8240-8247.
30. Ling X, Li F: Silencing of antiapoptotic survivin gene by multiple approaches of RNA interference technology. Biotechniques 2004, 36:450-460.
31. Bohula E A, Salisbury A J, Sohail M, Playford M P, Riedemann J, Southern E M, Macaulay V M: The efficacy of small interfering RNAs targeted to the type 1 insulin-like growth factor receptor (IGF1R) is influenced by secondary structure in the IGF1R transcript. J Biol Chem 2003, 278:15991-15997.
32. Paul C P, Good P D, Winer I, Engelke D R: Effective expression of small interfering RNA in human cells. Nat Biotechnol 2002, 20:505-508.
33. Yin J Q, Gao J, Shao R, Tian W N, Wang J, Wan Y: siRNA agents inhibit oncogene expression and attenuate human tumor cell growth. J Exp Ther Oncol 2003, 3:194-204.
34. Jackson A L, Bartz S R, Schelter J, Kobayashi S V, Burchard J, Mao M, Li B, Cavet G, Linsley P S: Expression profiling reveals off-target gene regulation by RNAi. Nat Biotechnol 2003, 21:635-637.
35. Gerke V, Moss S E: Annexins: from structure to function. Physiol Rev 2002, 82:331-371.
36. Makinen P I, Koponen J K, Karkkainen A M, Malm T M, Pulkkinen K H, Koistinaho J, Turunen M P, Yla-Herttuala S: Stable RNA interference: comparison of U6 and H1 promoters in endothelial cells and in mouse brain. J Gene Med 2006, 8:433-441.

What is claimed is:

1. An expression cassette for expressing a plurality of short hairpin (sh) RNAs, comprising
a plurality of promoters, at least one of which comprises two promoters in a bidirectional promoter in a back-to-back form; and
a plurality of nucleic acid sequences encoding said plurality of shRNAs,
wherein each of said plurality of promoters is operationally linked to one of said plurality of nucleic acid sequences encoding said plurality of shRNAs.

2. The expression cassette of claim 1, wherein said plurality of promoters comprises Pol III RNA promoters.

3. The expression cassette of claim 1, wherein said expression cassette further comprises linking sequences.

4. The expression cassette of claim 1, wherein said expression cassette further comprises restriction endonuclease cleavage sites.

5. A method of silencing mRNA in a cell, comprising the step of
introducing into said cell one or more expression cassettes for expressing a plurality of shRNAs, wherein said one or more expression cassettes comprises
a plurality of promoters, at least one of which comprises two promoters in a bidirectional promoter in a back-to-back form; and
a plurality of nucleic acid sequences encoding said plurality of shRNAs,
wherein each of said plurality of promoters is operationally linked to one of said plurality of nucleic acid sequences encoding said plurality of shRNAs.

6. A method of preparing an expression cassette for expressing a plurality of shRNAs, comprising the steps of
preparing a polymerase chain reaction (PCR) template containing at least one bidirectional promoter in a back-to-back form;
amplifying by PCR said PCR template using primers comprising nucleic acid sequences encoding said plurality of shRNAs, said step of amplifying producing an insert comprising
1) said at least one bidirectional promoter in a back-to-back form and
2) said nucleic acid sequences encoding said plurality of short hairpin RNAs; and
joining said insert to nucleic acid sequences encoding one or more additional promoters to form an expression cassette for expressing said plurality of shRNAs, wherein in said expression cassette, said promoters in said at least one bidirectional promoter in a back-to-back form and said one or more additional promoters are operationally linked to said nucleic acid sequences encoding said plurality of shRNAs.

7. The method of claim 6, wherein two promoters of said at least one bidirectional promoter in a back-to-back form have a 5′ overlap, and wherein said step of preparing is carried out by overlap PCR.

8. The method of claim 6, wherein said step of joining is carried out by ligation.

9. An expression cassette for expressing a plurality of short harpin (sh) RNAs, comprising:
a plurality of nucleic acid sequences encoding said plurality of shRNas;
at least one bidirectional promoter which includes two promoters in back-to-back form; and
at least two additional promoters for each of said at least one bidirectional promoter;
wherein said at least one bidirectional promoter and said at least two additional promoters are operationally linked to at least one of said plurality of nucleic acid sequences encoding said plurality of shRNAs.

10. A method of silencing mRNA in a cell, comprising the steps of:
a) introducing into said cell one or more expression cassettes for expressing a plurality of short hairpin (sh) RNAs, wherein each of said one or more expression cassettes comprises
i) a plurality of nucleic acid sequences encoding said plurality of shRNas,
ii) at least one bidirectional promoter which includes two promoters in back-to-back form; and
iii) at least two additional promoters for each of said at least one bidirectional promoter;
wherein said at least one bidirectional promoter and said at least two additional promoters are operationally linked to at least one of said plurality of nucleic acid sequences encoding said plurality of shRNAs; and
b) allowing for said plurality of shRNAs to be expressed from said one or more expression cassettes, said shRNAs silencing mRNA in said cell.


SEQUENCE LISTING (107 sequences)

SEQ ID NO: 1 - DNA, 30 bp, Artificial
  Synthetic oligonucleotide primer
cggatcgatccaaggtcgggcaggaagagg

SEQ ID NO: 2 - DNA, 18 bp, Artificial
  Synthetic oligonucleotide primer
ggtgtttcgtcctttcca

SEQ ID NO: 3 - DNA, 36 bp, Artificial
  Synthetic oligonucleotide primer
gaccttggatcgatccgaacgctgacgtcatcaacc

SEQ ID NO: 4 - DNA, 30 bp, Artificial
  Synthetic oligonucleotide primer
ggggatctgtgatctcatacagaacttata

SEQ ID NO: 5 - DNA, 17 bp, Artificial
  Synthetic 17 nucleotide overlap between hU6 and H1m promoters
gaccttggatcgatccg

SEQ ID NO: 6 - DNA, 11 bp, Artificial
  Synthetic 11 nucleotide loop
ggacagcacac

SEQ ID NO: 7 - DNA, 16 bp, Artificial
  Synthetic 18 nucleotide sequence complementary to hU6 promoter
ggtgtttcgtcctttc

SEQ ID NO: 8 - DNA, 14 bp, Artificial
  Synthetic nucleotide sequence complementary to 3′ end of H1m promoter
gggaaagagtgatc

SEQ ID NO: 9 - DNA, 11 bp, Artificial
  Synthetic linker sequence
gctcagcggag

SEQ ID NO: 10 - DNA, 11 bp, Artificial
  Synthetic linker sequence
ttcagtccgag

SEQ ID NO: 11 - DNA, 10 bp, Artificial
  Synthetic linker sequence
cttcctgtca

SEQ ID NO: 12 - DNA, 10 bp, Artificial
  Synthetic 10 nucleotide loop
ttgatatccg

SEQ ID NO: 13 - DNA, 19 bp, Artificial
  Synthetic Universal sequence containing Eco 31I restriction site
gcattcacggtctcatttg

SEQ ID NO: 14 - DNA, 19 bp, Artificial
  Synthetic universal sequence containing Eco 31I restriction site
gcagtaacggtctctcctc

SEQ ID NO: 15 - DNA, 32 bp, Artificial
  Synthetic oligonucleotide primer
caccgcggatcgatccgacgccgccatctcta

SEQ ID NO: 16 - DNA, 43 bp, Artificial
  Synthetic oligonucleotide primer
cttcgaagaattcccgggtctctcaaacaaggcttttctccaa

SEQ ID NO: 17 - DNA, 40 bp, Artificial
  Synthetic oligonucleotide primer
cttcgaaggtacctgcagtatttagcatgccccacccatc

SEQ ID NO: 18 - DNA, 38 bp, Artificial
  Synthetic oligonucleotide primer
ggaattcggtctctgaggtacccaggcggcgcacaagc

SEQ ID NO: 19 - DNA, 23 bp, Artificial
  Synthetic oligonucleotide primer
gcagcatcctagggaacctaaag

SEQ ID NO: 20 - DNA, 23 bp, Artificial
  Synthetic oligonucleotide primer
tgctcttgtattggcaatgtcaa

SEQ ID NO: 21 - DNA, 23 bp, Artificial
  Synthetic oligonucleotide primer
acctcaccaacccaaacactgta

SEQ ID NO: 22 - DNA, 23 bp, Artificial
  Synthetic oligonucleotide primer
acattctctcccgtttttgcact

SEQ ID NO: 23 - DNA, 22 bp, Artificial
  Synthetic oligonucleotide primer
agtgctcatggaaagggagttc

SEQ ID NO: 24 - DNA, 22 bp, Artificial
  Synthetic oligonucleotide primer
aaagctctggaagcccactttt

SEQ ID NO: 25 - DNA, 21 bp, Artificial
  Synthetic oligonucleotide primer
tgaatgaggccttggaactca

SEQ ID NO: 26 - DNA, 22 bp, Artificial
  Synthetic oligonucleotide primer
caggcccttctgtcttgaacat

SEQ ID NO: 27 - DNA, 20 bp, Artificial
  Synthetic oligonucleotide primer
cctaccgacctggtgtggaa

SEQ ID NO: 28 - DNA, 21 bp, Artificial
  Synthetic oligonucleotide primer
ctcgtcgtcctcaaccacagt

SEQ ID NO: 29 - DNA, 22 bp, Artificial
  Synthetic oligonucleotide primer
ggatgtctcctgagtccctcaa

SEQ ID NO: 30 - DNA, 21 bp, Artificial
  Synthetic oligonucleotide primer
aaggacttgctcgttggacaa

SEQ ID NO: 31 - DNA, 21 bp, Artificial
  Synthetic oligonucleotide primer
catgtgtgtggagagcgtcaa

SEQ ID NO: 32 - DNA, 21 bp, Artificial
  Synthetic oligonucleotide primer
ctacccagcctccgttatcct

SEQ ID NO: 33 - DNA, 23 bp, Artificial
  Synthetic oligonucleotide primer
aactccctcaagattgtcagcaa

SEQ ID NO: 34 - DNA, 23 bp, Artificial
  Synthetic oligonucleotide primer
cacagtcttctgagtggcagtga

SEQ ID NO: 35 - DNA, 23 bp, Artificial
  Synthetic oligonucleotide primer
aacagcctcaagatcatcagcaa

SEQ ID NO: 36 - DNA, 23 bp, Artificial
  Synthetic oligonucleotide primer
cacagtcttctgggtggcagtga

SEQ ID NO: 37 - DNA, 21 bp, Artificial
  Synthetic siRNA
aattctccgaacgtgtcacgt

SEQ ID NO: 38 - DNA, 21 bp, Artificial
  Synthetic siRNA
gacagctaggttatcacgatc

SEQ ID NO: 39 - DNA, 21 bp, Artificial
  Synthetic siRNA
tgcgttagctgcgtcaagcat

SEQ ID NO: 40 - DNA, 21 bp, Artificial
  Synthetic siRNA
acttactgtgcgtagttagcc

SEQ ID NO: 41 - DNA, 20 bp, Artificial
  Synthetic sequence encoding siRNA
gacatccgcaacgactatca

SEQ ID NO: 42 - DNA, 20 bp, Artificial
  Synthetic sequence encoding siRNA
gcccatgtgtgagaagacca

SEQ ID NO: 43 - DNA, 20 bp, Artificial
  Synthetic sequence encoding siRNA
accatcaacaatgagtacaa

SEQ ID NO: 44 - DNA, 20 bp, Artificial
  Synthetic sequence encoding siRNA
gagagcagagtggataacaa

SEQ ID NO: 45 - DNA, 21 bp, Artificial
  Synthetic sequence encoding siRNA
ctgtatctcagtggatcttca

SEQ ID NO: 46 - DNA, 21 bp, Artificial
  Synthetic sequence encoding siRNA
gagaattgagtctcctcattc

SEQ ID NO: 47 - DNA, 21 bp, Artificial
  Synthetic sequence encoding siRNA
ctgcctgagcaccataggtct

SEQ ID NO: 48 - DNA, 22 bp, Artificial
  Synthetic sequence encoding siRNA
aaccttaatgacagctcttaat

SEQ ID NO: 49 - DNA, 20 bp, Artificial
  Synthetic sequence encoding siRNA
ggtggagagagtgagacatt

SEQ ID NO: 50 - DNA, 21 bp, Artificial
  Synthetic sequence encoding siRNA
gcgtctgtgtgaacccatatc

SEQ ID NO: 51 - DNA, 21 bp, Artificial
  Synthetic sequence encoding siRNA
ggaattgatctctctggatta

SEQ ID NO: 52 - DNA, 21 bp, Artificial
  Synthetic sequence encoding siRNA
ggagtgcagttggagtgtaaa

SEQ ID NO: 53 - DNA, 21 bp, Artificial
  Synthetic sequence encoding siRNA
gtcttcactggttgttatgta

SEQ ID NO: 54 - DNA, 21 bp, Artificial
  Synthetic sequence encoding siRNA
gttaagtcacctgttacttag

SEQ ID NO: 55 - DNA, 21 bp, Artificial
  Synthetic sequence encoding siRNA
gcagagttgctctgcctgatg

SEQ ID NO: 56 - DNA, 21 bp, Artificial
  Synthetic sequence encoding siRNA
ctaatctgtgtgcatattgac

SEQ ID NO: 57 - DNA, 21 bp, Artificial
  Synthetic sequence encoding siRNA
ttatacactcggttaatctcc

SEQ ID NO: 58 - DNA, 21 bp, Artificial
  Synthetic sequence encoding siRNA
gacatcatctctgacacatct

SEQ ID NO: 59 - DNA, 21 bp, Artificial
  Synthetic sequence encoding siRNA
acaccaacttcgacgctgaga

SEQ ID NO: 60 - DNA, 20 bp, Artificial
  Synthetic sequence encoding siRNA
attgtcaacattctgactaa

SEQ ID NO: 61 - DNA, 21 bp, Artificial
  Synthetic sequence encoding siRNA
aatgcacagaggcaggacatt

SEQ ID NO: 62 - DNA, 21 bp, Artificial
  Synthetic sequence encoding siRNA
gtgcctatgggtcggtcaaac

SEQ ID NO: 63 - DNA, 21 bp, Artificial
  Synthetic sequence encoding siRNA
agagctacagtccttatgaca

SEQ ID NO: 64 - DNA, 21 bp, Artificial
  Synthetic sequence encoding siRNA
acattgaaacagcaatcaaga

SEQ ID NO: 65 - DNA, 21 bp, Artificial
  Synthetic sequence encoding siRNA
gcactggtgccgagtgcaaac

SEQ ID NO: 66 - DNA, 21 bp, Artificial
  Synthetic sequence encoding siRNA
gatcccttgcaactggaagaa

SEQ ID NO: 67 - DNA, 21 bp, Artificial
  Synthetic sequence encoding siRNA
ccatgaagactcagagatgta

SEQ ID NO: 68 - DNA, 21 bp, Artificial
  Synthetic sequence encoding siRNA
gcacaatgccgactgtcagaa

SEQ ID NO: 69 - DNA, 21 bp, Artificial
  Synthetic sequence encoding siRNA
atttatacctactgtaggctt

SEQ ID NO: 70 - DNA, 21 bp, Artificial
  Synthetic sequence encoding siRNA
gcaggatcagttaactattac

SEQ ID NO: 71 - DNA, 21 bp, Artificial
  Synthetic sequence encoding siRNA
ctgtagcttaagtacatgata

SEQ ID NO: 72 - DNA, 21 bp, Artificial
  Synthetic sequence encoding siRNA
actacttcccttattagatag

SEQ ID NO: 73 - DNA, 21 bp, Artificial
  Synthetic sequence encoding siRNA
ggaccaggtggtcgttaataa

SEQ ID NO: 74 - DNA, 21 bp, Artificial
  Synthetic sequence encoding siRNA
gtggattccgtactgttctac

SEQ ID NO: 75 - DNA, 21 bp, Artificial
  Synthetic sequence encoding siRNA
gaatgccgttcgccttcatta

SEQ ID NO: 76 - DNA, 21 bp, Artificial
  Synthetic sequence encoding siRNA
actgttggattgatccgaaac

SEQ ID NO: 77 - DNA, 21 bp, Artificial
  Synthetic sequence encoding siRNA
ggatgatctatcaccagaaga

SEQ ID NO: 78 - DNA, 19 bp, Artificial
  Synthetic sequence encoding siRNA
gaaggcatggaccaaataa

SEQ ID NO: 79 - DNA, 19 bp, Artificial
  Synthetic sequence encoding siRNA
ctaatgatgccagagaaga

SEQ ID NO: 80 - DNA, 20 bp, Artificial
  Synthetic sequence encoding siRNA
caagaatcgcattgacattg

SEQ ID NO: 81 - DNA, 21 bp, Artificial
  Synthetic sequence encoding siRNA
caccgtacaactactcttaca

SEQ ID NO: 82 - DNA, 21 bp, Artificial
  Synthetic sequence encoding siRNA
ggctgattgtcctcacacaat

SEQ ID NO: 83 - DNA, 21 bp, Artificial
  Synthetic sequence encoding siRNA
gaatttggtacaagaataatt

SEQ ID NO: 84 - DNA, 21 bp, Artificial
  Synthetic sequence encoding siRNA
gttacacggagctactataga

SEQ ID NO: 85 - DNA, 21 bp, Artificial
  Synthetic sequence encoding siRNA
gaaaccatgatgcttacattt

SEQ ID NO: 86 - DNA, 20 bp, Artificial
  Synthetic sequence encoding siRNA
ggaggacctgagagtgctca

SEQ ID NO: 87 - DNA, 21 bp, Artificial
  Synthetic sequence encoding siRNA
gtgggcttccagagctttcta

SEQ ID NO: 88 - DNA, 21 bp, Artificial
  Synthetic sequence encoding siRNA
ccttaggaaatgtgcaaataa

SEQ ID NO: 89 - DNA, 21 bp, Artificial
  Synthetic sequence encoding siRNA
gctacgacggctggtttaata

SEQ ID NO: 90 - DNA, 21 bp, Artificial
  Synthetic sequence encoding siRNA
gaacattgctctataccaatg

SEQ ID NO: 91 - DNA, 21 bp, Artificial
  Synthetic sequence encoding siRNA
acgcaagatgctactaaagaa

SEQ ID NO: 92 - DNA, 21 bp, Artificial
  Synthetic sequence encoding siRNA
cctcatgacatagcaagttat

SEQ ID NO: 93 - DNA, 21 bp, Artificial
  Synthetic sequence encoding siRNA
cagtaaggtggtgaatagact

SEQ ID NO: 94 - DNA, 21 bp, Artificial
  Synthetic sequence encoding siRNA
cgctacctcaacaatggactt

SEQ ID NO: 95 - DNA, 21 bp, Artificial
  Synthetic sequence encoding siRNA
gacgagctagcggaccacatt

SEQ ID NO: 96 - DNA, 21 bp, Artificial
  Synthetic sequence encoding siRNA
catctatggcaccaccgttta

SEQ ID NO: 97 - DNA, 21 bp, Artificial
  Synthetic sequence encoding siRNA
attgagctagcagtggtgaaa

SEQ ID NO: 98 - DNA, 21 bp, Artificial
  Synthetic sequence encoding siRNA
aggatgtatagtgttgatgga

SEQ ID NO: 99 - DNA, 21 bp, Artificial
  Synthetic sequence encoding siRNA
ccacaactatgcaagccatct

SEQ ID NO: 100 - DNA, 21 bp, Artificial
  Synthetic sequence encoding siRNA
gaatgattccgcgtctgtaat

SEQ ID NO: 101 - DNA, 21 bp, Artificial
  Synthetic sequence encoding siRNA
accacatgcttcgcaagaaga

SEQ ID NO: 102 - DNA, 21 bp, Artificial
  Synthetic sequence encoding siRNA
ggaaggctgggacgggaaatt

SEQ ID NO: 103 - DNA, 21 bp, Artificial
  Synthetic sequence encoding siRNA
ggaggctggtgatgaggatat

SEQ ID NO: 104 - DNA, 21 bp, Artificial
  Synthetic sequence encoding siRNA
atctagtgaacgctacgctta

SEQ ID NO: 105 - DNA, 17 bp, Artificial
  Synthetic linker sequence
gaccttggatcgatccg

SEQ ID NO: 106 - DNA, 11 bp, Artificial
  Synthetic linker sequence
gctcagcggag

SEQ ID NO: 107 - DNA, 11 bp, Artificial
  Synthetic linker sequence
ttcagtccgag